(19) United States
(12) Patent Application Publication
(10) Pub. No.: US 2021/0364519 A1
(43) Pub. Date: Nov. 25, 2021

(54) DETERMINING CANCER RESPONSIVENESS TO TREATMENT

(71) Applicant: Queensland University of Technology, Berisbane, Queensland (AU)

(72) Inventors: Joshua T. Burgess, Kelvin Grove, Queensland (AU); Emma Bolderson, Kelvin Grove, Queensland (AU); Derek J. Richard, Kelvin Grove, Queensland (AU); Kenneth J. O'Byrne, Kelvin Grove, Queensland (AU)

(21) Appl. No.: 17/053,707

(22) PCT Filed: Dec. 21, 2018
(86) PCT No.: PCT/AU2018/051393

§ 371 Date: Nov. 6, 2020

(30) Foreign Application Priority Data
May 8, 2018 (AU) 2018901563

(51) Int. Cl. G01N 33/574; G01N 33/50; A61K 45/06

(52) U.S. Cl. CPC G01N 33/57415; G01N 2333/4703; A61K 45/06; G01N 33/5041

(57) ABSTRACT

Provided herein are methods of treating a cancer with or predicting the responsiveness of a cancer to an anti-cancer agent which is capable of inhibiting an enzyme that mediates repair of DNA strand breaks, such as PARP, said method including the step of determining an expression level of SASH1. A method of treating a cancer that includes administering a therapeutically effective amount of an agent that increases the expression and/or an activity of SASH1 is also provided. Further provided herein are methods for identifying an agent that modulates the expression and/or an activity of SASH1 for use in the treatment of cancer.

### FIELD

THIS INVENTION relates to cancer. More particularly, this invention relates to methods of treating and/or determining the responsiveness to treatment and/or prognosis of cancers, in particular lung cancer and cancers of the reproductive system such as breast cancer.

### BACKGROUND

Breast cancer is the second most common cancer worldwide, comprising 25% of all female cancers. Owing to improved management strategies, the survival rate has increased in the past few decades. The disease, however, is still the second highest cause of cancer-associated deaths in women [1]. Current therapeutic strategies for breast cancer patients are based primarily on the pathological characteristics of the patient's tumour. Expression of hormone (oestrogen (ER) and progesterone) and human epidermal growth factor 2 (HER2) receptors is routinely assessed in diagnostic practice, as these proteins are currently the strongest prognostic and predictive biomarkers in breast cancer. Treatment options are most limited for tumours that are negative for these receptors (‘triple negative’) [2, 3]. Accordingly, there remains a need for the development of new therapies as well as prognostic markers for not only breast cancer, but cancer more broadly.

The poly(ADP-ribose) polymerase (PARP) inhibitors, such as Olaparib, were originally designed to treat BRCA mutated or triple negative breast cancer (loss of functional BRCA1 or 2 is often referred to as “BRACAness”), however BRCA1 and 2 have failed to be reliable biomarkers for sensitivity to these therapeutic agents. Nonetheless, “BRCAness” and Loss of Heterozygosity (LOH) currently remain the complimentary diagnostic for patient sensitivity to PARP inhibitors, but these readouts are not high confidence predictors of drug response. This has resulted in the requirement of large Phase III clinical trials to demonstrate survival/progression benefit. Despite this, numerous clinical trials with respect to the effectiveness of PARP inhibitors are being conducted in multiple solid cancer types including ovarian, prostate, breast and lung cancer. Accordingly, there remains a need for the development of an improved companion/complimentary diagnostics for PARP inhibitor treatment in cancer.

SAM and SH3 domain containing 1 (SASH1) was initially identified as a putative tumour suppressor gene, based on detection of significantly lower mRNA levels in breast, lung, thyroid and colorectal cancers compared to adjacent normal tissue [4, 5]. Low SASH1 expression also correlated with aggressive tumour growth, metastasis and poor prognosis in colon cancer [6]. As many as 74% of breast cancers show decreased SASH1 transcript expression [4]. This observation was later confirmed in an immunohistochemistry study that found reduced expression of SASH1 in breast cancer compared to adjacent normal tissue [7]. Methylation of the SASH1 promoter, particularly CpG_26.27 and CpG_54.55, correlates with SASH1 repression in breast cancer [7].

The exact functions of SASH1 in normal tissues and in cancer are still unclear, but the protein is known to be localised to the nucleus, and its SAM and SH3 domains imply signalling, adaptor and/or molecular scaffold functions [8, 9]. The suggested tumour suppressive role of SASH1 is consistent with studies demonstrating that depletion increased cellular viability, proliferation and migration in A549 lung cancer cells [5, 10-12], whilst overexpression resulted in a significant increase in apoptosis [5]. In addition, ectopic SASH1 expression has been shown to promote the expression of apoptotic proteins including Caspase 3 [10]. Apart from studies investigating expression, mutation status and DNA methylation, the role of SASH1 in breast cancer is poorly understood.

### SUMMARY

The present invention broadly relates to determining expression levels of SASH1 as a predictive and/or prognostic marker of the response of cancers to treatment with a PARP inhibitor. In some aspects, the invention also broadly relates to the treatment of cancer using agents that induce and/or increase SASH1 expression. In a particular form, the cancer is a cancer of the reproductive system such as breast cancer.

In a first aspect, the invention provides a method of predicting the responsiveness of a cancer to an anti-cancer agent in a subject, wherein the anti-cancer agent at least partly inhibits activity of an enzyme that mediates repair of a DNA strand break, said method including the step of determining an expression level of a SASH1 protein or encoding nucleic acid in one or a plurality of cancer cells, tissues or organs of the subject, wherein the expression level of the SASH1 protein or encoding nucleic acid indicates or correlates with relatively increased or decreased responsiveness of the cancer to the anti-cancer agent.

In particular embodiments, a relatively decreased level of SASH1 protein or encoding nucleic acid indicates or correlates with relatively increased responsiveness of the cancer to the anti-cancer agent; and/or a relatively increased level of SASH1 protein or encoding nucleic acid indicates or correlates with relatively decreased responsiveness of the cancer to the anti-cancer agent.

In one embodiment, the method of the present aspect includes the further step of treating the cancer in the subject.

In a second aspect, the invention provides a method of treating cancer in a subject, said method including the step of determining an expression level of SASH1 protein or encoding nucleic acid in one or a plurality of cancer cells, tissues or organs of the subject and based on the determination made, initiating, continuing, modifying or discontinuing a cancer treatment.

Suitably, the cancer treatment comprises the administration of a therapeutically effective amount of an anti-cancer agent that inhibits activity of an enzyme that mediates repair of a DNA strand break.

In certain embodiments, the cancer treatment comprises administering to the subject a therapeutically effective amount of an agent that inhibits or prevents the expression and/or an activity of SASH1.

In a third aspect, the invention provides a method of treating a cancer in a subject, including the step of administering to the subject a therapeutically effective amount of an agent that inhibits or prevents the expression and/or an activity of SASH1 in combination with an anti-cancer agent that inhibits activity of an enzyme that mediates repair of a DNA strand break.

Referring to the first, second and third aspects, the enzyme is suitably poly(ADP-ribose) polymerase (PARP).

In a fourth aspect, the invention provides a method of treating a cancer in a subject, including the step of administering to the subject a therapeutically effective amount of an agent that increases the expression and/or an activity of SASH1.

Suitably, the agent is a small organic molecule.

With respect to the first, second, third and fourth aspects, the anti-cancer agent or cancer treatment suitably is or comprises a PARP inhibitor. Preferably, the PARP inhibitor is selected from the group consisting of olaparib, veliparib, rucaparib, iniparib, talazoparib, niraparib, 3-aminobenzamide, ME0328, PJ34, AG-14361, INO-1001, UPF-1069, AZD-2461, CEP 9722, A-966492 and any combination thereof.

In a fifth aspect, the invention provides a method for identifying or producing an agent for use in the treatment of cancer in a subject including the steps of:

(a) contacting a cell that expresses a SASH1 nucleic acid or protein; with a candidate agent; and

(b) determining whether the candidate agent modulates the expression and/or an activity of SASH1.

In certain embodiments, the candidate agent, at least partly, reduces, eliminates, suppresses or inhibits the expression and/or the activity of SASH1. In alternative embodiments, the candidate agent, at least partly, increases the expression and/or the activity of SASH1.

Suitably, the agent is an antibody or a small organic molecule.

In a sixth aspect, the invention provides an agent identified or produced by the method of the fourth aspect.

In a seventh aspect, the invention provides a kit for predicting the responsiveness of a cancer to an anti-cancer agent in a subject, wherein the anti-cancer agent inhibits activity of an enzyme that mediates repair of a DNA strand break, the kit comprising at least one reagent capable of determining an expression level of a SASH1 protein or encoding nucleic acid in one or a plurality of cancer cells, tissues or organs of the subject, wherein the expression level of the SASH1 protein or encoding nucleic acid indicates or correlates with relatively increased or decreased responsiveness of the cancer to the anti-cancer agent.

In particular embodiments, a relatively decreased level of SASH1 protein or encoding nucleic acid indicates or correlates with relatively increased responsiveness of the cancer to the anti-cancer agent; and/or a relatively increased level of SASH1 protein or encoding nucleic acid indicates or correlates with relatively decreased responsiveness of the cancer to the anti-cancer agent.

Referring to the present aspect, the enzyme is suitably poly(ADP-ribose) polymerase (PARP). Accordingly, the anti-cancer agent suitably is or comprises a PARP inhibitor. Preferably, the PARP inhibitor is selected from the group consisting of olaparib, veliparib, rucaparib, iniparib, talazoparib, niraparib, 3-aminobenzamide, ME0328, PJ34, AG-14361, INO-1001, UPF-1069, AZD-2461, CEP 9722, A-966492 and any combination thereof.

Suitably, the present kit further includes a collection of data comprising correlation data between the expression level the SASH1 protein or encoding nucleic acid and responsiveness of the cancer to the anti-cancer agent.

Suitably, the collection of data is on a computer-readable medium.

In particular embodiments, the kit is for use in the method of the aforementioned aspects.

Suitably, the cancer of the aforementioned aspects is a cancer of the reproductive system. Preferably, the cancer of the reproductive system includes breast cancer, ovarian cancer, cervical cancer, uterine cancer, prostate cancer or testicular cancer. More preferably, the cancer of the reproductive system is breast cancer.

In other embodiments, the cancer of the aforementioned aspects is or comprises a lung cancer. Preferably, the lung cancer includes squamous cell carcinoma, adenocarcinoma, large cell carcinoma, small cell carcinoma and mesothelioma.

In an eighth aspect, the invention provides a method of determining a prognosis for a breast cancer in a subject, said method including the step of determining an expression level of SASH1 nucleic acid or protein in one or a plurality of cancer cells, tissues or organs of the subject, wherein an expression level of SASH1 indicates or correlates with a less or more favourable cancer prognosis for said breast cancer.

In particular embodiments, the breast cancer is ER positive (ER⁺) breast cancer or ER negative (ER−) breast cancer.

Suitably, the subject of the above aspects is a mammal, preferably a human.

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

The indefinite articles ‘a’ and ‘an’ are used here to refer to or encompass singular or plural elements or features and should not be taken as meaning or defining “one” or a “single” element or feature. For example, “a” cell includes one cell, one or more cells and a plurality of cells.

### BRIEF DESCRIPTION OF THE FIGURES

FIG. 1. Cleavage of SASH1 by Capase-3 is required for apoptosis. A) Recombinant Caspase-3 cleaves SASH1 between amino acids 230 and 231. B) Overexpression of SASH1 cleaved fragments indicates that cleavage of SASH1 is required for its apoptotic role.

FIG. 2. Overexpression of SASH1 or SASH1 cleaved C-terminus 231-1247 increase NF-κB nuclear protein levels. A) Quantification of NF-κB nuclear protein levels through immunofluorescence.

FIG. 3. SASH1 is required for Homologous Recombination and Genomic Stability. A) SASH1 protein levels accumulate following DNA damage stimulation. B) SASH1 is depletion significantly reduces Homologous recombination efficiency. C) SASH1 is required for the efficient clearance of DNA damage marker yH2AX. D) SASH1 depletion results in genomic instability measured via comet assay.

FIG. 4. SASH1 Protein levels correlate to Olaparib, Talazoparib and Veliparib Sensitivity. Correlation of PARP inhibitor Olaparib IC50 and SASH1 protein level in A) Breast, D) Lung and E) Ovarian cancer cell lines. B) Correlation of PARP inhibitor Talazoparib IC50 and SASH1 protein level in breast cancer cell lines. C) Correlation of PARP inhibitor Veliparib IC50 and SASH1 protein level in breast cancer cell lines.

FIG. 5. Depletion of SASH1 increases cancer cell sensitivity to Olaparib. A) Cell lines depleted of SASH1 have a reduced IC50 to Olaparib. B) Combined data of cell lines with SASH1 depletion showing a significant reduction in IC50 to Olaparib.

FIG. 6. Over expression of SASH1 infers resistance to Olaparib in U2OS cells.

FIG. 7. There is no Correlation between SASH1 and known PARP inhibitor sensitivity markers BRCA1 or BRCA2. Correlation of mRNA expression of SASH1 and A) BRCA1 or B) BRCA2. Online Data base COXPRES.

FIG. 8. SASH1 expression is associated with relapse and survival in breast cancer. (A) Kaplan Meier analysis of the relationships between SASH1 mRNA (left) or protein (right) expression and clinical outcomes in ER-positive and ER-negative breast cancer. BCSS, breast cancer-specific survival. Log-rank p values and hazard ratios (HR; 95% confidence intervals in parentheses) are indicated. (B) Representative SASH1 IHC images of breast cancer tissue microarray cores. Two grade-3 (G3) invasive ductal carcinomas (IDC) with negative and strongly positive nuclear SASH1 expression are shown at low and high magnification. (C,D) SASH1 stratification of BCSS in ER+ disease showed similar trends in subgroups with both high and low proliferative indices according to Ki67 expression (C) or mitotic score (D). Proportions of SASH1-high and -low cases were not different in ER+ subgroups with high or low proliferative indices (X2=chi square test).

FIG. 9. SASH1 protein expression in breast cancer cell lines. Breast cancer cell lines were analysed for expression of SASH1 by immunoblotting. Representative immunoblot is shown in (A), and (B) shows densitometric quantification of SASH1 expression relative to β actin. Data shown are means+/−standard deviation from three independent experiments, arbitrarily normalised to MCF7.

FIG. 10. Ectopic SASH1 expression increases cell death. (A) Confirmation of SASH1 overexpression by immunoblotting. Breast cancer cell lines were transfected with expression constructs encoding a PCMV6-SASH1-GFP fusion protein or PCMV6-GFP alone, then harvested after 48 h for lysate preparation and SASH1/(3-actin immunoblotting. OE, overexpression (B) SASH1 overexpression increases breast cancer cell line death. Cell lines were transfected as above, then stained with Hoechst 33342 and propidium iodide (PI) after 48 h and quantified using digital fluorescent microscopy. Data shown are the mean relative proportions of GFP-positive, PI-positive (dead and late apoptotic) cells +/−standard deviation from three independent experiments. Differences between SASH1-GFP and GFP control cultures were assessed using two-tailed t-tests. *p<0.05, **p<0.005.

FIG. 11. Chloropyramine increases SASH1 expression in breast cancer cell lines. (A-H) Cells were treated with 25 or 50 μM chloropyramine for 24 h, then lysates were prepared and SASH1 protein expression was analysed using immunoblotting. Immunoblot band intensities were quantified relative to b-actin in three independent experiments. The reproducibility and significance of changes in SASH1 expression with treatment were assessed using two-tailed t tests. *p<0.05, **p<0.005.

FIG. 12. Chloropyramine induces dose-dependent reduction of breast cancer cell line growth that involves apoptosis. (A-H) Changes in adherent breast cancer cell line confluence following chloropyramine treatment. Cells were treated with chloropyramine for 96 h and imaged using light microscopy and digitally analysed to assess confluence relative to an untreated control culture. (I-K) Chloropyramine induces apoptosis in breast cancer cell lines. Cells were stained with propidium iodide and an Annexin V-FITC antibody conjugate 48 h post-chloropyramine treatment, and analysed by flow cytometry. All data shown are means+/−the standard deviation from three independent experiments. Statistical analysis was performed using two-tailed t-tests; *p<0.05, **p<0.005, ***p<0.0005.

FIG. 13. SASH1 depletion partially rescues chloropyramine-induced apoptosis in breast cancer cell lines. (A) Cells were transduced with negative control or SASH1 esiRNAs. After 72 h, cell lysates were prepared and SASH1 expression was analysed relative to β-actin by immunoblotting. KD, knockdown. (B-D) Cells were transduced as above, and chloropyramine was added 24 h post-transfection. Cultures were imaged by light microscopy and digitally analysed to assess confluence relative to the untreated control at 96 h post treatment. Data shown are means+/−the standard deviation from three independent experiments. t-tests were used to compare cell confluence with and without SASH1 depletion at each of the chloropyramine doses; *p<0.05.

FIG. 14. (a) SASH1 protein levels in BRCA deficient and BRCA proficient tumours; (b) Correlation of SASH1 levels with responsiveness to treatment with rucaparib.

### DETAILED DESCRIPTION

The present invention is at least partly predicated on the surprising discovery that SASH1 is a predictive biomarker of PARP inhibitor treatment in cancer. Additionally, the invention described herein is predicated on the discovery that modulating, and more particularly, increasing SASH1 expression may be an effective anti-cancer treatment. Further, the present invention is at least partly predicated on the discovery that SASH1 is a prognostic marker in cancer of the reproductive systems such as breast cancer, as well as other solid tumours, such as lung and gastric cancer.

In one particular aspect, the present invention resides in a method of predicting the responsiveness of a cancer to an anti-cancer agent in a subject, wherein the anti-cancer agent inhibits activity of an enzyme that mediates repair of a DNA strand break, said method including the step of determining an expression level of SASH1 nucleic acid or encoded protein in one or a plurality of cancer cells, tissues or organs of the subject, wherein an altered or modulated expression level of SASH1 nucleic acid or encoded protein indicates or correlates with relatively increased or decreased responsiveness of the cancer to the anti-cancer agent.

It would be understood by the skilled artisan that the SASH1 gene comprises a nucleotide sequence that encodes the protein SAM and SH3 domain-containing protein 1. Other names for SASH1 may include Proline-Glutamate Repeat-Containing Protein 3, 2500002E12Rik, DJ323M4.1, KIAA0790, DJ323M4, SH3D6A and PEPE1. Non-limiting examples of Accession Numbers referencing the nucleotide sequence of the SASH1 gene, or its encoded protein, as are well understood in the art, in humans include NM_015278 and NP_056093.3. As generally used herein, “SASH1” may refer to a SASH1 nucleic acid or encoded protein, unless otherwise specified.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

As used herein a “gene” is a nucleic acid which is a structural, genetic unit of a genome that may include one or more amino acid-encoding nucleotide sequences and one or more non-coding nucleotide sequences inclusive of promoters and other 5′ untranslated sequences, introns, polyadenylation sequences and other 3′ untranslated sequences, although without limitation thereto. In most cellular organisms a gene is a nucleic acid that comprises double-stranded DNA.

The term “nucleic acid” as used herein designates single- or double-stranded DNA and RNA. DNA includes genomic DNA and cDNA. RNA includes mRNA, RNA, RNAi, siRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as inosine, methylycytosine, methylinosine, methyladenosine and/or thiouridine, although without limitation thereto.

Also included are, “variant” nucleic acids that include nucleic acids that comprise nucleotide sequences of naturally occurring (e.g., allelic) variants and orthologs (e.g., from a different species) of SASH1. Preferably, nucleic acid variants share at least 70% or 75%, preferably at least 80% or 85% or more preferably at least 90%, 91%, 92%, 93%, 94%, 95%, 96%, 97%, 98% or 99% sequence identity with a nucleotide sequence disclosed herein.

Also included are nucleic acid fragments. A “fragment” is a segment, domain, portion or region of a nucleic acid, which respectively constitutes less than 100% of the nucleotide sequence. A non-limiting example is an amplification product or a primer or probe. In particular embodiments, a nucleic acid fragment may comprise, for example, at least 10, 15, 20, 25, 30 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000 and 7500 contiguous nucleotides of said nucleic acid.

As used herein, a “polynucleotide” is a nucleic acid having eighty (80) or more contiguous nucleotides, while an “oligonucleotide” has less than eighty (80) contiguous nucleotides. A “probe” may be a single or double-stranded oligonucleotide or polynucleotide, suitably labelled for the purpose of detecting complementary sequences in Northern or Southern blotting, for example. A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. A “template” nucleic acid is a nucleic acid subjected to nucleic acid amplification.

By “protein” is meant an amino acid polymer. The amino acids may be natural or non-natural amino acids, D- or L-amino acids as are well understood in the art. As would be appreciated by the skilled person, the term “protein” also includes within its scope phosphorylated forms of a protein (i.e., a phosphoprotein) and/or glycosylated forms of a protein (i.e. a glycoprotein). A “peptide” is a protein having no more than fifty (50) amino acids. A “polypeptide” is a protein having more than fifty (50) amino acids.

Also provided are protein “variants” such as naturally occurring (e.g. allelic variants) and orthologs of SASH1. Preferably, protein variants share at least 70% or 75%, preferably at least 80% or 85% or more preferably at least 90%, 91%, 92%, 93%, 94%, 95%, 96%, 97%, 98% or 99% sequence identity with an amino acid sequence of SASH1 disclosed herein or known in the art.

Also provided are protein fragments, inclusive of peptide fragments that comprise less than 100% of an entire amino acid sequence. In particular embodiments, a protein fragment may comprise, for example, at least 10, 15, 20, 25, 30 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 1000, 1050, 1100, 1150 and 1200 contiguous amino acids of said protein.

As generally used herein, the terms “cancer”, “tumour”, “malignant” and “malignancy” refer to diseases or conditions, or to cells or tissues associated with the diseases or conditions, characterized by aberrant or abnormal cell proliferation, differentiation and/or migration often accompanied by an aberrant or abnormal molecular phenotype that includes one or more genetic mutations or other genetic changes associated with oncogenesis, expression of tumour markers, loss of tumour suppressor expression or activity and/or aberrant or abnormal cell surface marker expression.

Cancers may include any aggressive or potentially aggressive cancers, tumours or other malignancies such as listed in the NCI Cancer Index at http://www.cancer.gov/cancertopics/alphalist, including all major cancer forms such as sarcomas, carcinomas, lymphomas, leukaemias and blastomas, although without limitation thereto. These may include breast cancer, lung cancer inclusive of lung adenocarcinoma, cancers of the reproductive system inclusive of ovarian cancer, cervical cancer, uterine cancer and prostate cancer, cancers of the brain and nervous system, head and neck cancers, gastrointestinal cancers inclusive of colon cancer, colorectal cancer and gastric cancer, liver cancer, kidney cancer, skin cancers such as melanoma and skin carcinomas, blood cell cancers inclusive of lymphoid cancers and myelomonocytic cancers, cancers of the endocrine system such as pancreatic cancer and pituitary cancers, musculoskeletal cancers inclusive of bone and soft tissue cancers, although without limitation thereto.

With respect to the present and hereinafter described aspects, the cancer is suitably a cancer of the reproductive system, such as breast cancer, ovarian cancer, cervical cancer, uterine cancer, prostate cancer or testicular cancer. Preferably, the cancer of the reproductive system is breast cancer. To this end, the skilled person would appreciate that breast cancer may include any aggressive breast cancers and cancer subtypes known in the art, such as triple negative breast cancer, grade 2 breast cancer, grade 3 breast cancer, lymph node positive (LN+) breast cancer, HER2 positive (HER2+) breast cancer, ER negative (ER⁻) breast cancer and ER positive (ER+) breast cancer.

In other embodiments, the cancer of the aspects disclosed herein is, or comprises, a lung cancer. To this end, it would be apparent that lung cancer may include any aggressive lung cancers and cancer subtypes known in the art, such as non-small cell carcinoma (i.e., squamous cell carcinoma, adenocarcinoma and large cell carcinoma), small cell carcinoma and mesothelioma.

The term “a DNA strand break” includes where a single-strand of a DNA duplex is cleaved or broken (single-strand breaks), and where both strands of a DNA duplex are cleaved or broken (double-strand breaks). Such DNA strand breaks may be caused by extrinsic factors, such as ionizing radiation, ultraviolet rays, agents, or other mutagens which are present in food or the environment, or intrinsic factors such as an active oxygen, which is generated in the process of metabolism, and error(s) upon DNA replication.

In particular embodiments, the cancer has a reduced or impaired ability or capacity for repairing DNA strand breaks and/or demonstrates an increased susceptibility or incidence of DNA strand breaks. In this regard, cancer cells may demonstrate defects in DNA strand break repair or an increased incidence thereof, which contributes, at least in part, to the aberrant growth, differentiation and/or migration properties thereof. Accordingly, such cancer cells may become particularly susceptible to the induction of DNA strand breaks therein. Some cancer cells acquire defects in one or more particular DNA strand break repair pathways or mechanisms and subsequently become dependent on a compensatory mechanism in order to survive. Hence, targeted inhibition of this compensatory mechanism in combination with induction of DNA damage can selectively kill cancer cells but spare their normal counterparts (i.e., synthetic lethality).

With respect to the present aspect, the enzyme that mediates repair of said DNA strand break may be any known in the art. Preferably, the enzyme is poly(ADP-ribose) polymerase (PARP).

Poly (ADP-ribose) polymerase (PARP) is a family of proteins generally involved in a number of cellular processes, and more particularly, DNA repair and programmed cell death. The PARP family includes approximately 18 proteins (e.g., PARP1, PARP2, VPARP (PARP4), Tankyrase-1 and -2 (PARP-5a or TNK5, and PARP-5b or TNKS2), PARP3, PARP6, TIPARP (or “PARP7”), PARP8, PARP9, PARP10, PARP11, PARP12, PARP14, PARP15, and PARP16), which display a certain level of homology in their catalytic domain but typically differ in their cellular functions. PARP-1 and PARP-2 are considered unique members of the family, in that their catalytic activities are stimulated by the occurrence of DNA strand breaks. In this regard, PARP has been implicated in the signalling of DNA damage through its ability to recognize and rapidly bind to DNA single or double strand breaks.

In relation to the above, the anti-cancer agent suitably is or comprises a PARP inhibitor. The term “PARP inhibitor” as generally used herein refers to an inhibitor or antagonist of poly(ADP-ribose) polymerase activity. In particular embodiments, the PARP inhibitor specifically inhibits a particular PARP protein or proteins, such as PARP1 and/or PARP2. It would be apparent to the skilled artisan that when a PARP inhibitor is administered to a subject the PARP activity within the subject is altered, and more preferably reduced. A drug able to decrease the expression level of one or more PARPs expression is also considered a PARP inhibitor. In one embodiment, a prodrug of a PARP inhibitor is administered to a subject that is converted to the compound in vivo where it inhibits PARP.

The PARP inhibitor may be any type of compound. For example, the compound may be a small organic molecule or a biological compound such as an antibody or an enzyme. To this end, a person skilled in the art may be able to determine whether a compound is capable of inhibiting PARP activity and/or expression by any means known in the art. Exemplary assays for evaluating PARP activity and/or inhibition thereof include, for example, dot blots and BER assays that measure the direct activity of PARP to form poly ADP-ribose chains for example by using radioactive assays with tritiated substrate NAD or specific antibodies to the polymer chains formed by PARP activity.

It will be appreciated that the PARP inhibitor may be any known in the art, such as olaparib, veliparib, rucaparib, iniparib, talazoparib, niraparib, 3-aminobenzamide, ME0328, PJ34, AG-14361, INO-1001, UPF-1069, AZD-2461, CEP 9722, A-966492 and any combination thereof.

As would be understood by the skilled person, the expression level of a SASH1 nucleic acid or encoded protein may be relatively (i) higher, increased or greater; or (ii) lower, decreased or reduced when compared to an expression level in a control or reference sample, or to a threshold expression level. In one embodiment, an expression level may be classified as higher increased or greater if it exceeds a mean and/or median expression level of a reference population. In one embodiment an expression level may be classified as lower, decreased or reduced if it is less than the mean and/or median expression level of the reference population. In this regard, a reference population may be a group of subjects who have the same cancer type, subgroup, stage and/or grade as said mammal for which the expression level is determined.

Terms such as “higher”, “increased” and “greater” as used herein refer to an elevated amount or level of a SASH1 nucleic acid or protein, such as in a biological sample, when compared to a control or reference level or amount. The expression level of the SASH1 nucleic acid or protein may be relative or absolute. In some embodiments, the expression of the SASH1 nucleic acid or protein is higher, increased or greater if its level of expression is more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400% or at least about 500% above the level of expression of a SASH1 nucleic acid or protein in a control or reference level or amount.

The terms, “lower”, “reduced” and “decreased”, as used herein refer to a lower amount or level of the SASH1 nucleic acid or protein, such as in a biological sample, when compared to a control or reference level or amount. The expression level of the SASH1 nucleic acid or protein may be relative or absolute. In some embodiments, the expression of the SASH1 nucleic acid or protein is lower, reduced or decreased if its level of expression is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level or amount of expression of the SASH1 nucleic acid or protein in a control or reference level or amount.

The term “control sample” typically refers to a biological sample from a (healthy) non-diseased individual not having cancer. In one embodiment, the control sample may be from a subject known to be free of cancer. Alternatively, the control sample may be from a subject in remission from cancer. The control sample may be a pooled, average or an individual sample. An internal control is a marker from the same biological sample being tested.

As used herein, an expression level may be an absolute or relative amount of an expressed nucleic acid or protein. Accordingly, in some embodiments, the expression level of the SASH1 gene and/or a product thereof is compared to a control level of expression, such as the level of gene and/or protein expression of one or a plurality of “housekeeping” genes and/or proteins in one or more cancer cells, tissues or organs of the mammal.

In further embodiments, the expression level of the SASH1 nucleic acid or encoded protein is compared to a threshold level of expression, such as a level of gene and/or protein expression in non-cancerous tissue. A threshold level of expression is generally a quantified level of expression of SASH1. Typically, an expression level of SASH1 in a sample that exceeds or falls below the threshold level of expression is predictive of a particular disease state or outcome. The nature and numerical value (if any) of the threshold level of expression will typically vary based on the method chosen to determine the expression of the one or more genes, or products thereof, used in determining, for example, a prognosis and/or a response to anticancer therapy (e.g., PARP inhibitor therapy), in the mammal.

A person of skill in the art would be capable of determining the threshold level of SASH1 nucleic acid or protein expression in a sample that may be used in determining, for example, a prognosis and/or a response to anticancer therapy, using any method of measuring gene or protein expression known in the art, such as those described herein. In one embodiment, the threshold level is a mean and/or median expression level (median or absolute) of SASH1 in a reference population, that, for example, have the same cancer type, subgroup, stage and/or grade as said mammal for which the expression level is determined. Additionally, the concept of a threshold level of expression should not be limited to a single value or result. In this regard, a threshold level of expression may encompass multiple threshold expression levels that could signify, for example, a high, medium, or low probability of, for example, response to PARP inhibitor therapy.

In one embodiment, a lower expression level of SASH1 nucleic acid or encoded protein indicates or correlates with relatively increased responsiveness of the cancer to the anti-cancer treatment. In alternative embodiments, a lower expression level of SASH1 nucleic acid or encoded protein indicates or correlates with relatively decreased responsiveness of the cancer to the anti-cancer treatment.

In one particularly preferred embodiment, wherein the anti-cancer treatment is or comprises a PARP inhibitor, a relatively lower expression level of SASH1 nucleic acid or encoded protein indicates or correlates with relatively increased responsiveness of the cancer to the PARP inhibitor. Similarly, a relatively higher expression level of SASH1 nucleic acid or encoded protein suitably indicates or correlates with a relatively reduced responsiveness of the cancer to the PARP inhibitor.

Suitably, a reduced or decreased level of SASH1 indicates or correlates with relatively increased responsiveness of the cancer to the anticancer agent. Conversely, an increased level of SASH1 may indicate or correlate with relatively decreased responsiveness of the cancer to the anti-cancer agent. In particular embodiments, a decreased level of SASH1 indicates or correlates with relatively increased responsiveness of the cancer to the PARP inhibitor and/or an increased level of SASH1 indicates or correlates with relatively decreased responsiveness of the cancer to the PARP inhibitor. In this regard, the SASH1 expression levels are useful in the prediction of sensitivity and/or resistance of the subject's cancer to PARP inhibitor therapy.

The terms “determining”, “measuring”, “evaluating”, “assessing” and “assaying” are used interchangeably herein and may include any form of measurement known in the art, such as those described hereinafter.

Determining, assessing, evaluating, assaying or measuring nucleic acids of SASH1, such as RNA, mRNA and cDNA, may be performed by any technique known in the art. These may be techniques that include nucleic acid sequence amplification, nucleic acid hybridization, nucleotide sequencing, mass spectroscopy and combinations of any these.

Nucleic acid amplification techniques typically include repeated cycles of annealing one or more primers to a “template” nucleotide sequence under appropriate conditions and using a polymerase to synthesize a nucleotide sequence complementary to the target, thereby “amplifying” the target nucleotide sequence. Nucleic acid amplification techniques are well known to the skilled addressee, and include but are not limited to polymerase chain reaction (PCR); strand displacement amplification (SDA); rolling circle replication (RCR); nucleic acid sequence-based amplification (NASBA), Q-β replicase amplification; helicase-dependent amplification (HAD); loop-mediated isothermal amplification (LAMP); nicking enzyme amplification reaction (NEAR) and recombinase polymerase amplification (RPA), although without limitation thereto. As generally used herein, an “amplification product” refers to a nucleic acid product generated by a nucleic acid amplification technique.

PCR includes quantitative and semi-quantitative PCR, real-time PCR, allele-specific PCR, methylation-specific PCR, asymmetric PCR, nested PCR, multiplex PCR, touch-down PCR, digital PCR and other variations and modifications to “basic” PCR amplification.

Nucleic acid amplification techniques may be performed using DNA or RNA extracted, isolated or otherwise obtained from a cell or tissue source. In other embodiments, nucleic acid amplification may be performed directly on appropriately treated cell or tissue samples.

Nucleic acid hybridization typically includes hybridizing a nucleotide sequence, typically in the form of a probe, to a target nucleotide sequence under appropriate conditions, whereby the hybridized probe-target nucleotide sequence is subsequently detected. Non-limiting examples include Northern blotting, slot-blotting, in situ hybridization and fluorescence resonance energy transfer (FRET) detection, although without limitation thereto. Nucleic acid hybridization may be performed using DNA or RNA extracted, isolated, amplified or otherwise obtained from a cell or tissue source or directly on appropriately treated cell or tissue samples.

It will also be appreciated that a combination of nucleic acid amplification and nucleic acid hybridization may be utilized.

Determining, assessing, evaluating, assaying or measuring protein levels of SASH1 may be performed by any technique known in the art that is capable of detecting cell- or tissue-expressed proteins whether on the cell surface or intracellularly expressed, or proteins that are isolated, extracted or otherwise obtained from the cell of tissue source. These techniques include antibody-based detection that uses one or more antibodies which bind the protein, electrophoresis, isoelectric focussing, protein sequencing, chromatographic techniques and mass spectroscopy and combinations of these, although without limitation thereto. Antibody-based detection may include flow cytometry using fluorescently-labelled antibodies that bind SASH1, ELISA, immunoblotting, immunoprecipitation, in situ hybridization, immunohistochemistry and immuncytochemistry, although without limitation thereto. Suitable techniques may be adapted for high throughput and/or rapid analysis such as using protein arrays such as a TissueMicroArray™ (TMA), MSD MultiArrays' and multiwell ELISA, although without limitation thereto.

It will be appreciated that determining the expression of SASH1 may include determining both the nucleic acid levels thereof, such as by nucleic acid amplification and/or nucleic acid hybridization, and the protein levels thereof.

In certain embodiments, a gene expression level of SASH1 may be assessed indirectly by the measurement of a non-coding RNA, such as miRNA, that regulate gene expression. MicroRNAs (miRNAs or miRs) are post-transcriptional regulators that bind to complementary sequences in the 3′ untranslated regions (3′ UTRs) of target mRNA transcripts, usually resulting in gene silencing. miRNAs are short RNA molecules, on average only 22 nucleotides long. The human genome may encode over 1000 miRNAs, which may target about 60% of mammalian genes and are abundant in many human cell types. Each miRNA may alter the expression of hundreds of individual mRNAs. In particular, miRNAs may have multiple roles in negative regulation (e.g., transcript degradation and sequestering, translational suppression) and/or positive regulation (e.g., transcriptional and translational activation). Additionally, aberrant miRNA expression has been implicated in various types of cancer.

Further aspects of the invention relate to treatment of cancer in a subject.

In one particular aspect, the cancer treatment is performed in conjunction with determining an expression level of SASH1 protein or encoding nucleic acid in one or a plurality of cancer cells, tissues or organs of the subject, and based on the determination made, initiating, continuing, modifying or discontinuing the cancer treatment. In this regard, it would be appreciated that those methods described herein for predicting the responsiveness of a cancer to an anti-cancer agent, such as a PARP inhibitor, may further include the step of administering to the mammal a therapeutically effective amount of the anti-cancer agent. In a preferred embodiment, the anticancer treatment is administered when the SASH1 expression level indicates or correlates with relatively increased responsiveness of the cancer to the anti-cancer agent.

In certain embodiments, the cancer treatment comprises administering to a subject a therapeutically effective amount of an anti-cancer agent that inhibits activity of an enzyme that mediates repair of a DNA strand break.

In particular embodiments, the cancer treatment comprises administering to a subject a therapeutically effective amount of an agent that inhibits or prevents the expression and/or an activity of SASH1.

In another particular aspect, the invention provides a method of treating a cancer in a subject, including the step of administering to the subject a therapeutically effective amount of an agent that increases the expression and/or an activity of SASH1.

Suitably, the agent is a small organic molecule. A non-limiting example of a small organic molecule is chloropyramine.

In a particular aspect, the invention provides a method of treating a cancer in a subject, including the step of administering to the subject a therapeutically effective amount of an agent that inhibits or prevents the expression and/or an activity of SASH1 in combination with an anti-cancer agent that inhibits activity of an enzyme that mediates repair of a DNA strand break.

Suitably, the enzyme is poly(ADP-ribose) polymerase (PARP). Accordingly, the anti-cancer agent may be or comprise a PARP inhibitor, such as that previously described herein.

As noted earlier, particular cancers may demonstrate a reduced or impaired ability or capacity for repairing DNA strand breaks and/or possess an increased susceptibility to or incidence of DNA strand breaks potentially through acquired defects in one or more particular DNA strand break repair pathways. As a result, these cancer cells may become dependent on a compensatory mechanism, such as SASH1, in order to survive this ongoing DNA damage. Hence, targeted inhibition of SASH1 in combination with the induction of DNA strand breaks in the cancer by, for example, administration of a PARP inhibitor can be implemented to selectively kill cancer cells, as shown herein. To this end, the genetic interaction between PARP and SASH1 can be described as synthetic lethal. Synthetic lethality between two genes generally occurs where individual loss of either gene is compatible with life, but simultaneous loss of both genes results in cell death.

In particular embodiments, the agent that inhibits or prevents the expression and/or activity of SASH1, is administered (i) prior to; (ii) after; or (iii) simultaneously with, the administration of the anti-cancer agent. In one embodiment, administration of the agent that inhibits or prevents the expression and/or activity of SASH1, and the administration of the anti-cancer agent (either sequentially or concurrently) results in treatment or prevention of cancer that is greater than such treatment or prevention from administration of either the said agent or the anti-cancer agent in the absence of the other.

Suitably, the agent(s) is/are administered to a subject as a pharmaceutical composition comprising a pharmaceutically-acceptable carrier, diluent or excipient. In this regard, any dosage form and route of administration, such as those provided therein, may be employed for providing a subject with the composition of the invention.

Cancer treatments may include drug therapy, chemotherapy, antibody, nucleic acid and other biomolecular therapies, radiation therapy, surgery, nutritional therapy, relaxation or meditational therapy and other natural or holistic therapies, although without limitation thereto. Generally, drugs, biomolecules (e.g antibodies, inhibitory nucleic acids such as siRNA) or chemotherapeutic agents are referred to herein as “anti-cancer therapeutic agents” or “anti-cancer agents”.

Methods of treating cancer may be prophylactic, preventative or therapeutic and suitable for treatment of cancer in mammals, particularly humans. As used herein, “treating”, “treat” or “treatment” refers to a therapeutic intervention, course of action or protocol that at least ameliorates a symptom of cancer after the cancer and/or its symptoms have at least started to develop. As used herein, “preventing”, “prevent” or “prevention” refers to therapeutic intervention, course of action or protocol initiated prior to the onset of cancer and/or a symptom of cancer so as to prevent, inhibit or delay or development or progression of the cancer or the symptom.

The term “therapeutically effective amount” describes a quantity of a specified agent, such as a PARP inhibitor, sufficient to achieve a desired effect in a subject being treated with that agent. For example, this can be the amount of a composition comprising one or more agents that inhibit the activity of an enzyme that mediates repair of a DNA strand break described herein, necessary to reduce, alleviate and/or prevent a cancer or cancer associated disease, disorder or condition. In some embodiments, a “therapeutically effective amount” is sufficient to reduce or eliminate a symptom of a cancer. In other embodiments, a “therapeutically effective amount” is an amount sufficient to achieve a desired biological effect, for example an amount that is effective to decrease or prevent cancer growth and/or metastasis.

Ideally, a therapeutically effective amount of an agent is an amount sufficient to induce the desired result without causing a substantial cytotoxic effect in the subject. The effective amount of an agent useful for reducing, alleviating and/or preventing a cancer will be dependent on the subject being treated, the type and severity of any associated disease, disorder and/or condition (e.g., the number and location of any associated metastases), and the manner of administration of the therapeutic composition.

Suitably, the anti-cancer therapeutic agent is administered to a mammal as a pharmaceutical composition comprising a pharmaceutically-acceptable carrier, diluent or excipient.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, liposomes and other lipid-based carriers, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991), which is incorporated herein by reference.

Any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection is appropriate, for example, for administration of immunotherapeutic compositions, proteinaceous vaccines and nucleic acid vaccines.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

In particular embodiments, the anti-cancer therapy may be directed at inhibiting the action of and/or decreasing the expression of SASH1. In other embodiments, the anti-cancer therapy may be directed at promoting the action of and/or increasing the activity and/or expression of SASH1.

In alternative embodiments, the anti-cancer treatment may be directed at genes or gene products other than SASH. By way of example, the anti-cancer treatment may target genes or gene products that are known to interact, directly or indirectly, with SASH1 and/or modulate the expression of SASH1.

Accordingly, in certain embodiments, the cancer treatment comprises the administration of an anti-cancer agent that inhibits activity of an enzyme, such as poly(ADP-ribose) polymerase (PARP), that mediates repair of a DNA strand break, such as hereinbefore described. Preferably, the anti-cancer agent is or comprises a PARP inhibitor, such as that previously described herein.

In a particular embodiment, the invention provides a “companion diagnostic” with respect to the cancer treatment, whereby the expression level of SASH1 provides information to a clinician or the like that is used for the safe and/or effective administration of said cancer treatment.

Suitably, the cancer is of a type hereinbefore described, albeit without limitation thereto. Preferably, the cancer demonstrates an altered or modulated expression level of SASH1.

In yet another aspect, the invention provides a method for identifying an agent for use in the treatment of a cancer in a subject including the steps of:

(a) contacting a cell that expresses SASH1 nucleic acid or encoded protein with a candidate agent; and

(b) determining whether the candidate agent modulates the expression and/or an activity of SASH1.

In particular embodiments, the candidate agent, at least partly, reduces, eliminates, suppresses or inhibits the expression and/or the activity of SASH1. Given the present disclosure, it would be apparent that such agents may be used in combination with an anti-cancer agent that inhibits activity of an enzyme that mediates repair of a DNA strand break, such as a PARP inhibitor provided herein.

In alternative embodiments, the candidate agent, at least partly, increases the expression and/or the activity of SASH1. To this end, the agent may be used in monotherapy or alternatively in combination with an additional anti-cancer agent, such as those described herein.

Suitably, the agent possesses or displays little or no significant off-target and/or nonspecific effects.

Preferably, the agent is an antibody or a small organic molecule.

In embodiments relating to antibody inhibitors, the antibody may be polyclonal or monoclonal, native or recombinant. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

Generally, antibodies of the invention bind to or conjugate with an isolated protein, fragment, variant, or derivative of the protein product of SASH1. For example, the antibodies may be polyclonal antibodies. Such antibodies may be prepared for example by injecting an isolated protein, fragment, variant or derivative of the SASH1 protein product into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, supra, and in Harlow & Lane, 1988, supra.

Monoclonal antibodies may be produced using the standard method as for example, described in an article by Köhler & Milstein, 1975, Nature 256, 495, which to is herein incorporated by reference, or by more recent modifications thereof as for example, described in Coligan et al., CURRENT PROTOCOLS IN IMMUNOLOGY, supra by immortalizing spleen or other antibody producing cells derived from a production species which has been inoculated with one or more of the isolated SASH1 protein products and/or fragments, variants and/or derivatives thereof.

Typically, the inhibitory activity of candidate inhibitor antibodies may be assessed by in vitro and/or in vivo assays that detect or measure the expression levels and/or activity of the SASH1 protein in the presence of the antibody.

In embodiments relating to small organic molecule inhibitors, this may involve screening of large compound libraries, numbering hundreds of thousands to millions of candidate inhibitors (chemical compounds including synthetic, small organic molecules or natural products, such as inhibitory peptides or proteins) which may be screened or tested for biological activity at any one of hundreds of molecular targets in order to find potential new drugs, or lead compounds. Screening methods may include, but are not limited to, computer-based (“in silico”) screening and high throughput screening based on in vitro assays.

Typically, the active compounds, or “hits”, from this initial screening process are then tested sequentially through a series of other in vitro and/or in vivo tests to further characterize the active compounds. A progressively smaller number of the “successful” compounds at each stage are selected for subsequent testing, eventually leading to one or more drug candidates being selected to proceed to being tested in human clinical trials.

At the clinical level, screening a candidate agent may include obtaining samples from test subjects before and after the subjects have been exposed to a test compound. The levels in the samples of SASH1 protein may then be measured and analysed to determine whether the levels and/or activity of the SASH1 protein change after exposure to a candidate agent. By way of example, protein product levels in the samples may be determined by mass spectrometry, western blot, ELISA and/or by any other appropriate means known to one of skill in the art. Additionally, the activity of the protein products, such as their anti-apoptotic activity, may be determined by any method known in the art. This may include, for example, apoptosis assays, such as caspase activation and/or cleavage, annexin V positivity, chromatin morphology, extracellular phosphatidylserine and DNA fragmentation assays.

It would be appreciated that subjects who have been treated with candidate agents may be routinely examined for any physiological effects which may result from the treatment. In particular, the candidate agents will be evaluated for their ability to decrease cancer likelihood or occurrence in a subject. Alternatively, if the candidate agents are administered to subjects who have previously been diagnosed with cancer, they will be screened for their ability to slow or stop the progression of the cancer as well as induce disease remission.

In this regard, candidate agents that are identified of being capable of reducing, eliminating, suppressing or inhibiting the expression level and/or activity of SASH1 may then be administered to patients who are suffering from or are at risk of developing cancer. For example, the administration of a candidate agent which inhibits or decreases the activity and/or expression of SASH1 may treat the cancer and/or decrease the risk of cancer, if the increased activity of the biomarker is responsible, at least in part, for the progression and/or onset of the cancer.

In a related aspect, the invention provides an anti-cancer agent produced or identified by the aforementioned aspect.

In yet another aspect, the invention provides a kit for predicting the responsiveness of a cancer to an anti-cancer agent in a subject, wherein the anti-cancer agent inhibits activity of an enzyme that mediates repair of a DNA strand break, the kit comprising at least one reagent capable of determining an expression level of a SASH1 protein or encoding nucleic acid in one or a plurality of cancer cells, tissues or organs of the subject, wherein the expression level of the SASH1 protein or encoding nucleic acid indicates or correlates with relatively increased or decreased responsiveness of the cancer to the anti-cancer agent.

In particular embodiments, a relatively decreased level of SASH1 protein or encoding nucleic acid indicates or correlates with relatively increased responsiveness of the cancer to the anti-cancer agent; and/or a relatively increased level of SASH1 protein or encoding nucleic acid indicates or correlates with relatively decreased responsiveness of the cancer to the anti-cancer agent.

Referring to the present aspect, the enzyme is suitably poly(ADP-ribose) polymerase (PARP). Accordingly, the anti-cancer agent suitably is or comprises a PARP inhibitor. Preferably, the PARP inhibitor is selected from the group consisting of olaparib, veliparib, rucaparib, iniparib, talazoparib, niraparib, 3-aminobenzamide, ME0328, PJ34, AG-14361, INO-1001, UPF-1069, AZD-2461, CEP 9722, A-966492 and any combination thereof.

Suitably, the present kit further includes reference data for correlating the expression level the SASH1 protein or encoding nucleic acid and responsiveness of the cancer to the anti-cancer agent.

In particular embodiments, the reference data is on a computer-readable medium (e.g., software embodying or utilised by any one or more of the methodologies or functions described herein). The computer-readable medium can be included on a storage device, such as a computer memory (e.g., hard disk drives or solid state drives) and preferably comprises computer readable code components that when selectively executed by a processor implements one or more aspects of the present invention.

In a particular embodiment, the present kit provides a “companion diagnostic” whereby information with respect to SASH1 expression levels are utilised by a clinician or similar for the safe and effective administration of the anti-cancer agent.

Suitably, the present kit is for use in the method of the aforementioned aspects.

In a further broad aspect, the invention provides a method of determining a prognosis for a breast cancer in a subject, said method including the step of determining an expression level of SASH1 nucleic acid or protein in one or a plurality of cancer cells, tissues or organs of the subject, wherein an expression level of SASH1 indicates or correlates with a less or more favourable cancer prognosis for said breast cancer.

Suitably, the breast cancer of this aspect is ER positive (ER⁺) breast cancer or ER negative (ER−) breast cancer.

The terms “prognosis” and “prognostic” are used herein to include making a prognosis, which can provide for predicting a clinical outcome (with or without medical treatment), selecting an appropriate course of treatment (or whether treatment would be effective) and/or monitoring a current treatment and potentially changing the treatment. This may be at least partly based on determining expression levels of SASH1, which may be in combination with or addition to determining the expression levels of additional protein and/or other nucleic acid biomarkers. A prognosis may also include a prediction, forecast or anticipation of any lasting or permanent physical or psychological effects of cancer suffered by the subject after the cancer has been successfully treated or otherwise resolved. Furthermore, prognosis may include one or more of determining metastatic potential or occurrence, therapeutic responsiveness, implementing appropriate treatment regimes, determining the probability, likelihood to or potential for cancer recurrence after therapy and prediction of development of resistance to established therapies (e.g., chemotherapy). It would be appreciated that a positive prognosis typically refers to a beneficial clinical outcome or outlook, such as long-term survival without recurrence of the subject's cancer, whereas a negative prognosis typically refers to a negative clinical outcome or outlook, such as cancer recurrence or progression.

Suitably, the method of the present aspect further includes the step of diagnosing said subject as having a less favourable prognosis or a more favourable prognosis. In one embodiment, a relative or absolute decrease in the expression of SASH1 is diagnostic of a less favourable or poor prognosis in the subject. In a further embodiment, wherein the breast cancer is ER⁺ breast cancer, a relative or absolute increase in the expression of SASH1 is diagnostic of a more favourable prognosis in the subject. In another embodiment, wherein the breast cancer is ER⁻ breast cancer, a relative or absolute increase in the expression of SASH1 is diagnostic of a less favourable or poor prognosis in the subject.

With respect to the above, a cancer may have a relatively poor prognosis due to one or more of a combination of features or factors including: at least partial resistance to therapies available for cancer treatment; invasiveness; metastatic potential; recurrence after treatment; and a low probability of patient survival, although without limitation thereto. In particular embodiments, SASH1 expression levels are prognostic for aggressive disease, and in particular a shorter time to biochemical recurrence and/or a shorter patient survival time. In further embodiments, SASH1 expression levels correlate with or indicate metastatic cancer.

It will also be understood that a SASH1 expression level may be used to identify those poorer prognosis patients, such as those with larger and/or higher grade tumours, who may benefit from one or more additional anti-cancer therapeutic agents to the typical or standard anti-cancer treatment regime for that particular patient group.

It will be appreciated from the foregoing that the invention provides methods that determine a cancer prognosis for a patient and/or predict the responsiveness of a cancer to an anti-cancer treatment. Particular, broad embodiments of the invention include the step of treating the patient following determining a cancer prognosis and/or predicting the responsiveness of the cancer to an anti-cancer treatment. Accordingly, these embodiments relate to using information obtained about the cancer prognosis and/or the predicted responsiveness of the cancer to anti-cancer treatment to thereby construct and implement an anti-cancer treatment regime for the patient. In a preferred embodiment, this is personalized to a particular patient so that the treatment regime is optimized for that particular patient.

With respect to the aforementioned aspects, the term “subject” includes but is not limited to mammals inclusive of humans, performance animals (such as horses, camels, greyhounds), livestock (such as cows, sheep, horses) and companion animals (such as cats and dogs). Preferably, the subject is a human.

So that preferred embodiments of the invention may be fully understood and put into practical effect, reference is made to the following non-limiting examples.

### Example 1

The putative tumour suppressor SASH1 has been implicated in apoptosis and cellular proliferation. Work by the inventors has also indicated that SASH1 is also required for the repair of genetic breaks via the homologous recombination DNA repair pathway. PARP inhibitors are a new generation of therapeutics designed to kill cancer cells with defects in the homologous recombination pathway, via a process known as synthetic lethality. The most developed drug to date is Olaparib. Since our data implicates SASH1 in the homologous recombination pathway we sought to determine if low SASH1 expressing cells (that would have decreased homologous recombination activity) are sensitive to PARP1 inhibitors.

### Results

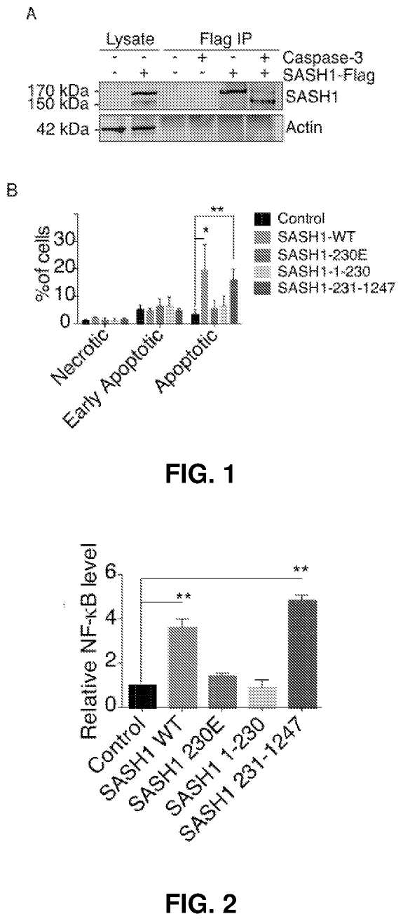

Current work by the present inventors has revealed that SASH1 has a prominent role in apoptosis. SASH1 is cleaved by the apoptotic regulator Caspase-3, with this cleavage event being required for a normal apoptotic response (FIG. 1). Furthermore Chloropyramine, a drug we identified by connectivity mapping, up-regulates SASH1 and enhances apoptosis in a SASH1 and UV dependent manner (see Example 2).

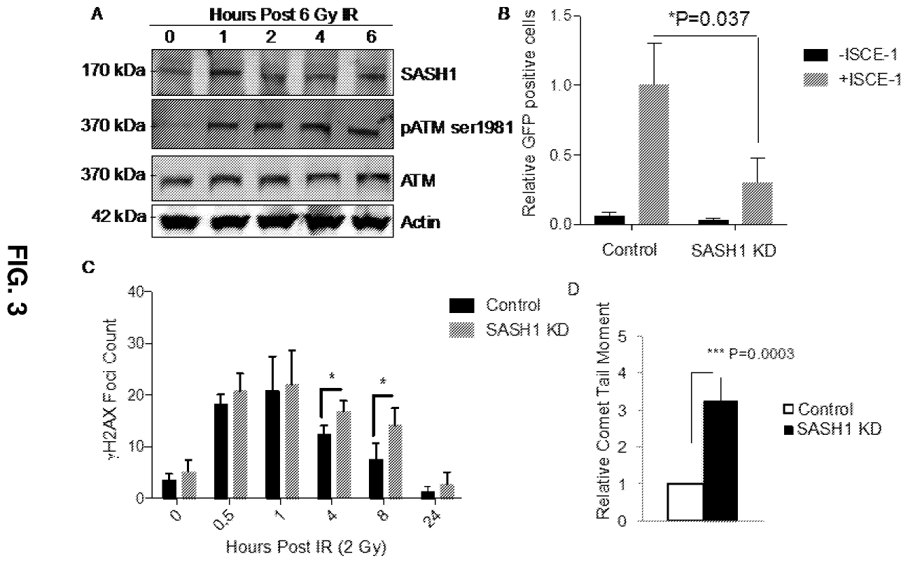

NF-κB has a central role to play in the induction of apoptosis following UV exposure, with the p65 subunit of NF-κB translocating from the cytoplasm to the nucleus after UV exposure. Our data demonstrates that SASH1 cleavage by Caspase-3 is required for the transport of the p65 subunit of NF-κB into the nucleus, with the ectopic expression of the cleaved SASH1 form being sufficient to allow NF-κB transport to the nucleus (FIG. 2).

SASH1 was identified as a gene that was inversely regulated with hSSB1. hSSB1 functions in the repair of double strand DNA breaks by homologous recombination. To assess if SASH1 was also required like hSSB1 and BRCA1/2 in the repair of double strand breaks a number of assays were undertaken.

The response of SASH1 to DNA damage from ionising radiation (IR) was assessed (FIG. 3A). The response observed was similar to that of other DNA damage response proteins, with stabilisation of protein levels. We next examined the ability of SASH1 depleted cells to repair double strand DNA breaks by homologous recombination using the GFP HR reporter cell line MCF7DRGFP. This indicated homologous recombination activity was reduced by loss of SASH1 (FIG. 3 B). This impairment is similar to that observed in BRCA1 & 2 depleted cells. The epigenetic marker γH2AX is an indirect measurement of double strand DNA breaks, therefore the repair of DNA damage can be assessed through γH2AX foci counts. Here it was found that cells depleted of SASH1 had a delay of γH2AX foci clearance following DNA damage induced by IR (FIG. 3 C). We next used a comet assay to assess the degree of genomic instability within SASH1 depleted cells, as compared to mock treated cells. SASH1 depletion resulted in a significantly (p=0.0003) longer comet tail indicating genomic instability was occurring in these depleted cells (FIG. 3D). These data, in combination, suggest that SASH1 is critical for DNA repair and the maintenance of genomic integrity through the homologous recombination pathway.

PARP proteins function in the detection and repair of single strand DNA breaks through the base excision repair pathway [9,10]. Therefore targeting PARP1 in tumours with deficient homologous recombination has been identified as a strategy to induce cancer cell death through apoptosis and necrosis. Use of these PARP inhibitors as a cancer treatment is being tested in clinical trials, with at least 53 studies (Table 1) currently being undertaken, commonly in combination with chemotherapeutics.

TABLE 1
Current clinical trails registered in the US for drugs targeting PARP1

| Drug | Company | Disease Target | Phase | Enrollments |
|---|---|---|---|---|
| Gemcitabine carboplatin | Sanofi | Squamous Cell Lung Cancer | Phase III | 780 |
| plus Iniparib | | | | |
| bsi-201 plus temozolomide | Sanofi | Glioblastoma | Phase I/II | 126 |
| [18F]FluorThanatrace PARP | Washington University | Cancer | Phase 0 | 50 |
| enzyme activity | School of Medicine | | | |
| Cyclophosphamide/Veliparib | National Cancer Institute (NCI) | Locally Advanced or Metastatic | Phase I | 55 |
| | | Breast Cancer | | |
| KU-0059436 (AZD2281)(PARP inhibitor) | AstraZeneca | Ovarian Neoplasms\|BRCA1 | Phase I | 95 |
| | | Protein\|BRCA2 Protein | | |
| Iniparib, gemcitabine and cisplatin | Sanofi | Non-small Cell Lung Cancer Stage IV | Phase I/II | 119 |
| JPI-289 | Jeil Pharmaceutical Co., Ltd. | Stroke | Phase I | 40 |
| MM-398 and Veliparib | National Cancer Institute (NCI) | Solid Tumors | Phase I | 48 |
| Cisplatin and Veliparib | National Cancer Institute (NCI) | Stage IV Breast Cancer BRCA1 Mutant | Phase II | 235 |
| Temozolomide and Veliparib | National Cancer Institute (NCI) | Stage IV Head and Neck Cancer | Phase I/II | 110 |
| Floxuridine and Veliparib | National Cancer Institute (NCI) | Stage IV Fallopian Tube Cancer\|Stage | Phase I | 102 |
| | | IV Ovarian Cancer\|Stage IV Primary | | |
| | | Peritoneal Cancer | | |
| Combination Chemotherapy With or | National Cancer Institute (NCI) | Head and Neck Cancer Stage IV | Phase I/II | 110 |
| Without Veliparib | | | | |
| Cisplatin, Etoposide and Veliparib | National Cancer Institute (NCI) | Extensive Stage Small Cell | Phase I/II | 168 |
| | | Lung Cancer | | |
| Temozolomide With or Without Veliparib | National Cancer Institute (NCI) | Relapsed or Refractory Small | Phase II | 110 |
| | | Cell Lung Cancer | | |
| veliparib with gemcitabine hydrochloride | National Cancer Institute (NCI) | Locally Advanced or Metastatic | Phase II | 107 |
| and cisplatin | | Pancreatic Cancer | | |
| Abiraterone Acetate and Prednisone | National Cancer Institute (NCI) | Metastatic Hormone-Resistant | Phase II | 148 |
| With or Without Veliparib | | Prostate Cancer | | |
| Veliparib | National Cancer Institute (NCI) | Persistent or Recurrent | Phase II | 51 |
| | | Epithelial Ovarian, Fallopian | | |
| | | Tube, or Primary Peritoneal Cancer | | |
| Veliparib, Radiation Therapy, and | National Cancer Institute (NCI) | Younger Patients With Newly | Phase I/II | 66 |
| Temozolomide | | Diagnosed Diffuse Pontine Gliomas | | |
| Veliparib, Pegylated Liposomal | National Cancer Institute (NCI) | Ovarian Cancer, Primary Peritoneal | Phase I | 48 |
| Doxorubicin Hydrochloride, | | Cancer, or Fallopian Tube Cancer | | |
| Carboplatin, and Bevacizumab | | | | |
| Veliparib and Dinacidib | National Cancer Institute (NCI) | Advanced Solid Tumors | Phase I | 130 |
| Veliparib With or Without Radiation | National Cancer Institute (NCI) | Stage III Non-small Cell Lung Cancer | Phase I/II | 162 |
| Therapy, Carboplatin, and Paclitaxel | | That Cannot Be Removed by Surgery | | |
| Veliparib, Paclitaxel, and | National Cancer Institute (NCI) | olid Tumors That Are Metastatic or | Phase I | 276 |
| Carboplatin | | Cannot Be Removed by Surgery and | | |
| | | Liver or Kidney Dysfunction | | |
| Veliparib, Bendamustine Hydrochloride, | National Cancer Institute (NCI) | Relapsed or Refractory Lymphoma, | Phase I/II | 41 |
| and Rituximab | | Multiple Myeloma, | | |
| | | or Solid Tumors | | |
| Paclitaxel, Cisplatin, and Veliparib | National Cancer Institute (NCI) | Advanced, Persistent, or Recurrent | Phase I/II | 66 |
| | | Cervical Cancer | | |
| Veliparib in Combination With | National Cancer Institute (NCI) | Locally Advanced or Metastatic | Phase I | 22 |
| Carboplatin and Paclitaxel | | Solid Tumors | | |
| Veliparib, Topotecan Hydrochloride, | National Cancer Institute (NCI) | Patients With Persistent or | phase II | 27 |
| and Filgrastim or Pegfilgrastim | | Recurrent Cervical Cancer | | |
| Veliparib and Radiation Therapy | National Cancer Institute (NCI) | Advanced Solid Malignancies | Phase I | 40 |
| | | With Peritoneal Carcinomatosis, | | |
| | | Epithelial Ovarian, Fallopian, | | |
| | | or Primary Peritoneal Cancer | | |
| Veliparib and Carboplatin | National Cancer Institute (NCI) | HER2-Negative Metastatic Breast Cancer | Phase I | 42 |
| ABT-888 and Gemcitabine Hydrochloride | National Cancer Institute (NCI) | Advanced Solid Tumors | Phase I | 31 |
| Veliparib With or Without Carboplatin | National Cancer Institute (NCI) | Stage III or Stage IV Breast Cancer | phase II | 71 |
| Veliparib and Pegylated Liposomal | National Cancer Institute (NCI) | Recurrent Ovarian Cancer, Fallopian | Phase I | 58 |
| Doxorubicin Hydrochloride | | Tube Cancer, or Primary Peritoneal | | |
| | | Cancer or Metastatic Breast Cancer | | |
| Veliparib and Temozolomide | National Cancer Institute (NCI) | Acute Leukemia | Phase I | 66 |
| Veliparib With or Without Mitomycin C | National Cancer Institute (NCI) | Unresectable, or Recurrent Solid Tumors | Phase I | 75 |
| Veliparib and Topotecan Hydrochloride | National Cancer Institute (NCI) | Solid Tumors, Relapsed or Refractory | Phase I/II | 102 |
| | | Ovarian Cancer, or Primary Peritoneal Cancer | | |
| Carboplatin, Paclitaxel, Bevacizumab, | National Cancer Institute (NCI) | Newly Diagnosed Stage II-IV Ovarian | Phase I | 474 |
| and Veliparib | | Epithelial, Fallopian Tube, or | | |
| | | Primary Peritoneal Cancer | | |
| Veliparib | National Cancer Institute (NCI) | Malignant Solid Tumors That Did Not | Phase I | 120 |
| | | Respond to Previous Therapy | | |
| Veliparib, Cyclophosphamide, and | National Cancer Institute (NCI) | Metastatic or Unresectable Solid Tumors | Phase I | 90 |
| Doxorubicin Hydrochloride | | or Non-Hodgkin Lymphoma | | |
| Veliparib and Topotecan With or Without | National Cancer Institute (NCI) | Relapsed or Refractory Acute Leukemia, | Phase I | 12 |
| Carboplatin | | High-Risk Myelodysplasia, or Aggressive | | |
| | | Myeloproliferative Disorders | | |
| Veliparib and Irinotecan Hydrochloride | National Cancer Institute (NCI) | Cancer That Is Metastatic or Cannot Be | Phase I | 48 |
| | | Removed by Surgery | | |
| Veliparib, Carboplatin, and Paclitaxel | National Cancer Institute (NCI) | Advanced Solid Cancer | Phase I | 107 |
| Rucaparib(CO-338; Formally Called | Cancer Research UK | Locally Advanced or Metastatic Breast | Phase II | 78 |
| AG-014699 or PF-0136738) | | Cancer or Advanced Ovarian Cancer | | |
| CEP-9722 and Temozolomide | Cephalon | Advanced Solid Tumors | Phase I | 26 |
| CEP-9722 in Combination With | Cephalon | Advanced Solid Tumors or Mantle | Phase I | 24 |
| Gemcitabine and Cisplatin | | Cell Lymphoma | | |
| SAR240550 (BSI-201) in Combination | Sanofi | Metastatic Triple Negative Breast Cancer | Phase II | 163 |
| With Gemcitabine/Carboplatin | | | | |
| SAR240550/Weekly Paclitaxel and | Sanofi | riple Negative Breast Cancer Patients | Phase II | 141 |
| Paclitaxel Alone | | | | |
| Enzalutamide and Niraparib | Paul Mathew, MD | Metastatic Castrate-Resistant | Phase I | 2 |
| | | Prostate Cancer | | |
| Olaparib | Yale University | HPV Positive and HPV Negative head | Phase I | 20 |
| | | and neck squamous cell carcinoma | | |
| Olaparib Priorto Surgery and | University Health | Ovarian, Primary Peritoneal, and | Phase II | 71 |
| Chemotherapy | Network, Toronto | Fallopian Tube Cancer (NEO) | | |
| Fluzoparib | Jiangsu HengRui Medicine | Advanced Solid Malignancies | Phase I | 42 |
| | Co., Ltd. | | | |
| [18F]FluorThanatrace Radiation | Abramson Cancer Center of the | Epithelial Ovarian, Fallopian Tube, | Phase I | 30 |
| | University of Pennsylvania | or Primary Peritoneal Cancer | | |
| Safety, Tolerability and | Jeil Pharmaceutical Co., Ltd. | Healthy Male Volunteers | Phase I | 24 |
| Pharmacokinetics/Pharmacodynamics | | | | |
| of JPI-289 | | | | |
| Niraparib and Temozolomide | Sarcoma Alliance for Research | Incurable Ewing Sarcoma | Phase I | 50 |
| or Irinotecan in Patients With | through Collaboration | | | |
| Previously Treated | | | | |
| Talazoparib Plus Irinotecan | St. Jude Children's Research | Refractory or Recurrent | Phase I | 60 |
| With or Without Temozolomide | Hospital | Solid Malignancies | | |

The justification of this approach is largely based on the presence of BRCA1 and/or BRCA2 mutations, or low expression of either gene, within the cancer cells. “BRCAness” has been described in solid tumours such as breast, prostate, ovarian and lung cancer. BRCA1/2 proteins are required for DNA double strand break repair via homologous recombination, the same pathway in which SASH1 functions. These mutations in BRCA1 & 2 often lead to reduced double strand break repair ability [11]. The inhibition of PARP1 results in unresolved single strand damage events, ultimately leading to double strand DNA breaks. BRCA1/2 deficient cells are unable to repair these double strand breaks, leading to the accumulation of DNA damage and subsequent cell death. This induction of cell death by PARP1 inhibitors in BRCA deficient cells is often referred to as synthetic lethality.

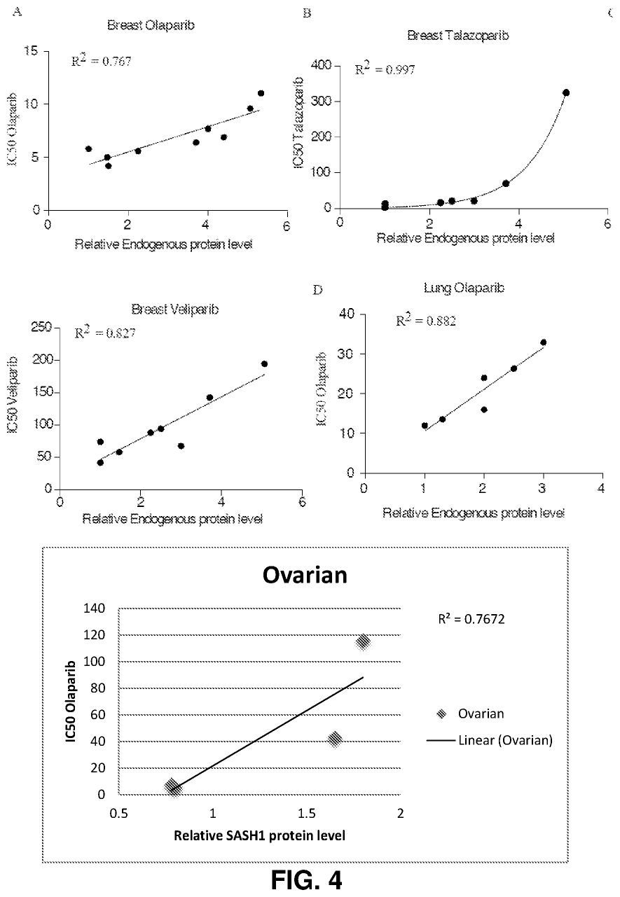

We further demonstrated that SASH1 protein levels correlate in a linear manner to cancer cell sensitivity to Olaparib (FIG. 4). The depletion of SASH1 using siRNA, resulted in an increase in sensitivity to Olaparib treatment in 9 out of 12 cell lines tested, consistent with the linear correlation between SASH1 protein levels and

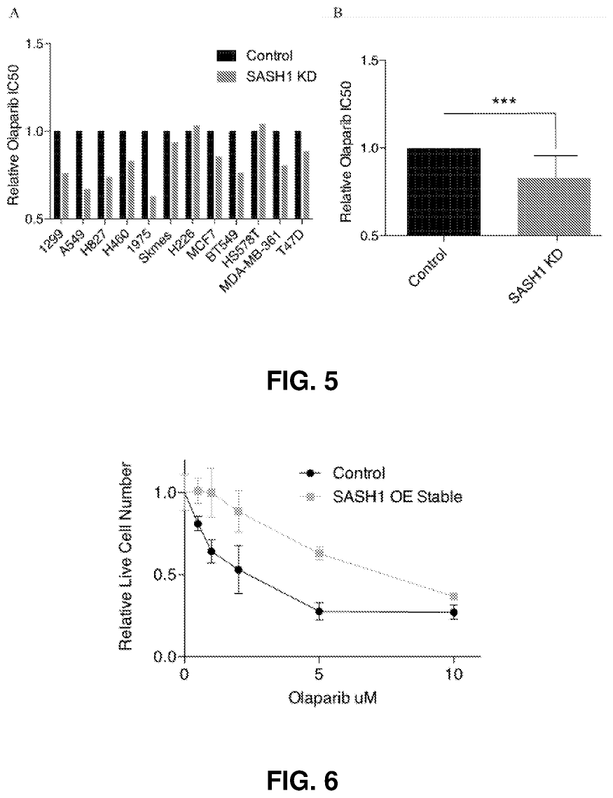

Olaparib sensitivity (FIG. 5).

Further to this, we made a stable U2OS cell line ectopically expressing recombinant SASH1. This demonstrated that over-expression of recombinant SASH1 in the U2OS cell line was sufficient to induce resistance to Olaparib (FIG. 6).

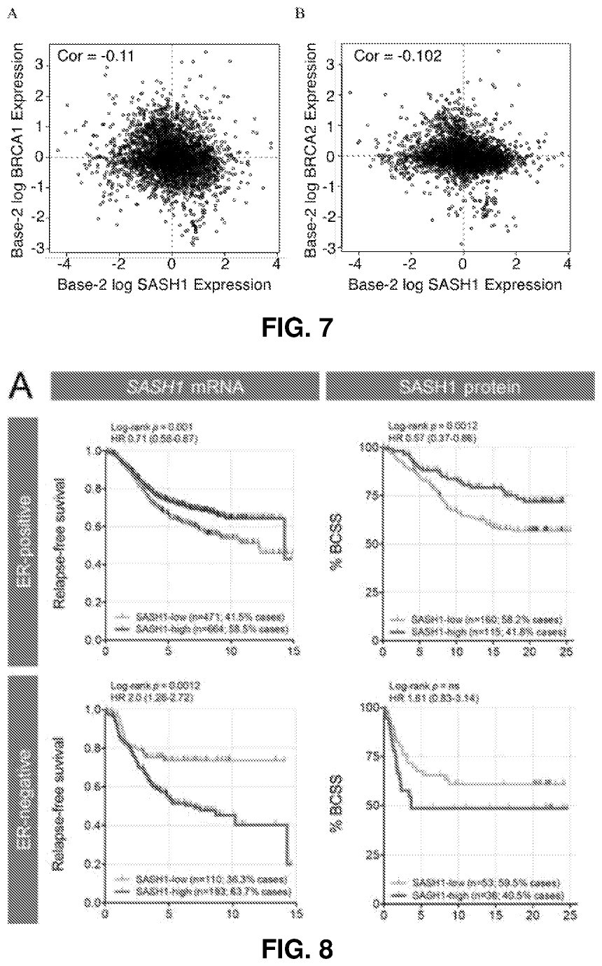

As the correlation of SASH1 and genomic stability is similar to that seen in BRCA1/2 depleted cells. Our group assessed if SASH1 mRNA levels correlated with BRCA1/2 transcript levels. On-line bioinformatic co-expression analysis of SASH1 and BRCA1/2 mRNA expression was undertaken (FIG. 7). There was no correlation between SASH1 and BRCA1/2 mRNA levels. This indicates that SASH1 is an independent marker of PARP inhibitor sensitivity. It should be noted however that mutational assessment of BRCA1/2 was not taken into consideration in this analysis.

### DISCUSSION

Our data clearly demonstrates that high SASH1 protein expression is associated with Olaparib resistance in both breast and lung cancer cell lines. In contrast low SASH1 protein levels were associated with sensitivity to PARP1 inhibition. Indeed SASH1 expression had a direct linear relationship with Olaparib sensitivity, with an R2 value of approximately 0.8 for breast cancer cell lines and 0.9 for lung cancer cell lines.

To determine if this was directly related to SASH1 protein levels we depleted SASH1 from the breast cancer cell lines using siRNA, this resulted in the sensitisation of all the cell lines to Olaparib, consistent with the correlation of SASH1 levels and Olaparib sensitivity. Inversely SASH1 overexpression in U2OS cells inferred resistance to Olaparib.

Olaparib was initially designed to treat BRCA1 or 2 deficient tumours, but BRCA1 and 2 have failed to be reliable biomarkers for sensitivity. Consistent with this, while SASH1 expression directly correlates with Olaparib sensitivity, SASH1 did not correlate with BRCA1 or BRCA2 expression, indicating that SASH1 may be a more robust marker of Olaparib sensitivity.

Our data presented herein identifies for the first time the molecular role of SASH1 in apoptosis. It further identifies that SASH1 tumour suppressor activity may not only be through the apoptosis pathway but also through the critical homologous recombination pathway. We demonstrate that SASH1 functions with the tumour suppressors, BRCA1 and BRCA2 in the homologous recombinational repair of cytotoxic double strand DNA breaks. As SASH1 appears to be required for homologous recombination we tested if we could induce synthetic lethality in low SASH1 expressing cells using the PARP1 inhibitor Olaparib. This indicated that there was an extremely strong linear relationship between SASH1 expression and Olaparib sensitivity with an R2 of approximately 0.8 in breast cancer cells lines and 0.9 in lung cancer cell lines.

### CONCLUSION

These data indicate that SASH1 levels in tumours are a predictive biomarker for the emerging novel DNA-repair targeted PARP1 inhibitors. The R2 values indicate, in cell line studies, that SASH1 is a robust marker that could have a large impact on the selection of patients for therapy with PARP1 inhibitors such as Olaparib. Furthermore, the cell line data provided herein indicates that olaparib sensitivity can be removed or induced by merely altering the level of this marker within cancer cells. Relevantly, there is currently no other effective reliable single gene/protein biomarker for PARP1 inhibitor sensitivity.

### REFERENCES

1. Zeller C, Hinzmann B, Seitz S, Prokoph H, Burkhard-Goettges E, Fischer J, et al. SASH1: a candidate tumor suppressor gene on chromosome 6q24.3 is downregulated in breast cancer. Oncogene. 2003rd ed. 2003; 22:2972-83.
2. Rimkus C, Martini M, Friederichs J, Rosenberg R, Doll D, Siewert J R, et al. Prognostic significance of downregulated expression of the candidate tumour suppressor gene SASH1 in colon cancer. Br. J. Cancer. 2006 ed. 2006; 95:1419-23.
3. Koch C A, Anderson D, Moran M F, Ellis C, Pawson T. SH2 and SH3 domains: elements that control interactions of cytoplasmic signaling proteins. Science. 1991st ed. 1991; 252:668-74.
4. Chen, Chen Y, Dong L L, Zhang J S. Effects of SASH1 on lung cancer cell proliferation, apoptosis, and invasion in vitro. Tumour Biol. 2012 ed. 2012.
5. Claudio J O, Zhu Y X, Benn S J, Shukla A H, McGlade C J, Falcioni N, et al. HACS1 encodes a novel SH3-SAM adaptor protein differentially expressed in normal and malignant hematopoietic cells. Oncogene. 2001st ed. 2001; 20:5373-7.
6. Meng Q, Zheng M, Liu H, Song C, Zhang W, Yan J, et al. SASH1 regulates proliferation, apoptosis, and invasion of osteosarcoma cell. Mol. Cell. Biochem. 2013; 373:201-10.
7. Lin S, Zhang J, Xu J, Wang H, Sang Q, Xing Q, et al. Effects of SASH1 on melanoma cell proliferation and apoptosis in vitro. Mol Med Rep. 2012; 6:1243-8.
8. Yang, Liu M, Gu Z, Chen J, Yan Y, Li J. Overexpression of SASH1 related to the decreased invasion ability of human glioma U251 cells. Tumour Biol. 2012 ed. 2012.
9. Marchetti C, Imperiale L, Gasparri M L, Palaia I, Pignata S, Boni T, et al. Olaparib, PARP1 inhibitor in ovarian cancer. Expert Opin Investig Drugs. 2012; 21:1575-84.
10. Wang Z, Wang F, Tang T, Guo C. The role of PARP1 in the DNA damage response and its application in tumor therapy. Front. Med. S P Higher Education Press; 2012; 6:156-64.
11. Savage K I, Harkin D P. BRCA1, a “complex” protein involved in the maintenance of genomic stability. FEBS J. 2015; 282:630-46.

### Example 2

In this study the relationships between SASH1 expression (both mRNA and protein) and breast cancer clinicopathologic parameters were analysed. Using in silico connectivity mapping and in vitro modelling, the antihistamine chloropyramine was identified as a drug that induces SASH1-dependent cell death in a panel of breast cancer cell lines, suggesting that further studies investigating potential clinical applications are warranted.

### Materials and Methods

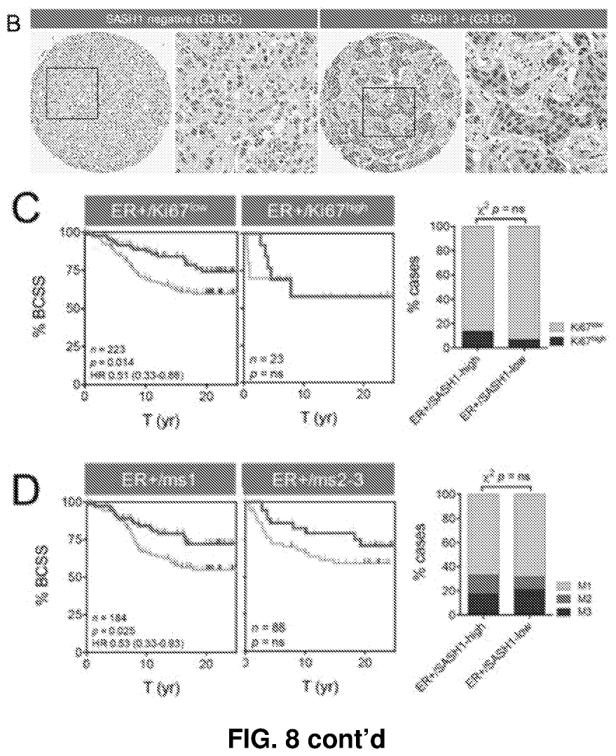

In silico analysis of SASH1 mRNA prognostic significance: The relationships between SASH1 mRNA expression, relapse-free and overall survival, as well as prognostic significance in a multivariate model, were analysed using the KM plotter breast cancer database [13]. Three different SASH1 array probes were analysed and representative data from the ‘JetSet’ optimal probe is presented [29] (FIG. 8).

Immunohistochemistry (IHC) and tissue microarray (TMA) analysis: SASH1 protein expression in breast cancer was investigated by IHC analysis of the Queensland breast cancer follow-up (QFU) resource, which comprises TMAs of 449 invasive breast carcinomas (sampled in duplicate) and associated clinical data, including survival outcomes of over 20 years [30]. The use of the patient data and clinical samples used in this study were approved by human research ethics committees of the University of Queensland and the Royal Brisbane and Women's Hospital (RBWH).

Four μm TMA sections were processed in a decloaker for antigen retrieval in EDTA buffer (pH 8.8) for 15 minutes, and then IHC was performed using an anti-SASH1 antibody (Sigma Prestige HPA029947; 1:850), and the Mach 1 Universal HRP-Polymer Detection kit (Biocare Medical). Haematoxylin-counterstained, mounted sections were then scanned at 40× magnification on an Aperio AT Turbo slide scanner (Leica Biosystems). Digital images of individual tissue cores were scored by a qualified Pathologist (AMM) according to tumour cell nuclear and cytoplasmic intensity, and proportion of tumour cells stained. Using the maximum score of duplicate tissue cores for each case, associations between SASH1 expression and clinicopathologic variables were investigated using chi-square and log-rank tests (GraphPad Prism v6).

Cell Culture and transfection: Breast cancer cell lines were cultured at 37° C. with 5% CO2 in RPMI with 10% FCS (MDA-MB-231, MDA-MB-361, T47-D, and BT-549), DMEM with 10% FCS (MCF7, MDA-MB-468, and Hs578T) or Ham's F12 with 5% FBS and 10 μg/ml recombinant human epidermal growth factor (SUM1315). Insulin was supplemented at 0.01 mg/mL for the MCF7, Hs578T, BT-549, T47-D, and SUM1315 cell lines. Cells were routinely passaged with trypsin and maintained at low passage. Chloropyramine (Sigma-Aldrich) was added to adherent cultured cells 24 hours after seeding at the indicated concentrations (0-100 μM). Cell validation was performed by Kerry Richard at QIMR Berghofer. SPR profiles matched against Children's oncology group cell culture and Xenograft repository (http://www.cogcell.org) STR genotyping data for cell-lines, February 2016.

For siRNA experiments, esiRNAs (Sigma) targeting SASH1 or non-specific control oligos were transfected using RNAiMax (Invitrogen) as per the manufacturer's instructions. Doubletransfections were performed 24 hours apart and samples were analysed 72 hours after the initial transfection where optimal SASH1 depletion was observed. For overexpression studies, the full-length SASH1 cDNA was cloned into the mammalian expression vector PCMV6 (Origene). Three μg of DNA (SASH1-GFP or GFP) and 6 μL of Lipofectamine 2000 (Invitrogen) were used to transfect cells in a T25 flask, as per the manufacturer's instructions. Cells were harvested 24-48 h post-transfection for optimal overexpression and death assessment as indicated in figure legends.

Immunoblotting: Immunoblotting was carried out as described previously [31]. Briefly, cells were lysed (20 mM Hepes pH 8.0, 150 mM KCl, 5% glycerol, 10 mM MgCl2, 0.5 mM EDTA, 0.02% NP-40, freshly supplemented with NaF, NaVO4, PMSF and protease inhibitors) and sonicated. Lysates were cleared by centrifugation and protein concentrations were estimated using the Bradford assay (Bio-Rad). Typically 50 μg of protein lysate was resolved on Bolt 4-12% gradient gels (Invitrogen) and proteins were transferred to nitrocellulose membrane (Bio-Rad). Membranes were blocked in 2% fish skin gelatin, 1% tween-20 in PBS (Sigma) for 1 h and incubated with primary antibodies overnight 4° C. in the same buffer. Following incubation with secondary antibodies, membranes were visualised using a Li—COR Odyssey infrared scanner. Fluorescence intensity was quantified relative to a loading control (β-actin or Histone H3) using Image J software.

Cell death assay: Following incubation of cells with the indicated treatments, Propidium iodide (10 μg/ml) and Hoechst (1 μg/ml) were added 30 min before imaging. Cells were imaged on an IN Cell Analyzer 2200 (GE Healthcare; 10× objective). Live/dead cell analysis was performed using InCell analysis software.

Cell confluency assay: Cells were seeded at 2,500 cells per well in 96 well plates (Nunc). Cells were allowed to adhere for 24 h before chloropyramine addition and then imaged every 2 hours for 96 hours in an IncuCyte ZOOM® live cell imager (Essen Bioscience) to calculate confluence.

Annexin V/Propidium Iodide (PI) analysis: Annexin V/PI staining was carried out as described [32]. Briefly, treated or untreated cells (adherent and floating) were harvested using trypsin and centrifugation, washed in PBS and then stained according to the Promega Annexin V-FITC apoptosis detection kit protocol. Annexin V-positive (apoptotic) cells were detected using a Gallios flow cytometer system and quantified with Flow Jo software.

Statistical analysis: Most statistical tests were performed using Graph Pad Prism V6. Associations between breast tumour SASH1 expression and clinicopathologic variables were investigated using chi square tests. Relationships between breast tumour SASH1 expression and relapse-free or overall survival were represented with Kaplan Meier curves and analysed using the log-rank test. Analysis of SASH1-mediated changes in apoptosis and proliferation, and SASH1 expression following chloropyramine treatment were investigated with students two-tailed t-tests. For multivariate analysis of SASH1 mRNA prognostic significance was performed using the parameters available (MKI67 and ERBB2 expression) and inbuilt function in the KM plotter database [13].

For multivariate analysis of SASH1 protein prognostic significance in ER+ breast cancer, we performed stepwise Cox regression analysis using MedCalc® software (v13.2) including HER2 status (determined by CISH according to diagnostic criteria), Ki67 status (nuclear staining in at least 20% tumour cells), histological grade (assessed by an experienced Pathologist (SRL) and tumour size (derived from clinical pathology reports). These data were complete for 223 ER+ cases. p Values >0.05 were considered significant.

Drug screen with Connectivity mapping: A gene expression connectivity mapping approach was employed to identify candidate compounds that may induce SASH1 expression. SASH1 was mapped to Affymetrix HG-U133A probeset IDs to form a query gene signature. This was compared to the reference drug expression profiles in the CMap02 database using the sscMap algorithm [33, 34]. Compounds with statistically significant positive connection to the query gene signature were selected as candidate SASH1 inducing drugs for further laboratory validation as described.

### Results

The prognostic significance of SASH1 expression was assessed at the mRNA level through a meta-analysis of publicly available breast cancer gene expression data with associated clinical follow-up information from the KM plotter database [13]. Kaplan-Meier analysis showed that over the whole patient cohort, loss of SASH1 mRNA expression was associated with a poor prognosis. However, segregation of the large KM plotter cohort into subgroups according to ER status showed that the prognostic significance of SASH1 mRNA was context-dependent. At 15 years post-diagnosis higher levels of SASH1 expression were associated with better outcome amongst patients with ER-positive breast cancer (p=0.001; HR 1.4 (1.15-1.73)), but considerably worse outcome in ER-negative disease (p=0.001; HR 0.5 (0.37-0.78)) (FIG. 8A and Table 2).

The relationship between SASH1 protein expression and clinicopathologic parameters was evaluated in a separate, clinically-annotated cohort of 379 invasive breast tumours by immunohistochemical (IHC) analysis of a tissue microarray (TMA) [14, 15]. Using a wellcharacterised anti-SASH1 antibody, predominantly nuclear staining of variable intensity was seen in breast tumour cells, the intensity being scored as negative, weakly/moderately positive and strongly positive (FIG. 8B). The percentage of tumour cell nuclei stained was reasonably homogeneous within individual tissue cores. Chi square analyses demonstrated that nuclear SASH1 expression was associated with ER expression, but none of the other clinicopathologic parameters available (Table 3; p=0.0035).

In agreement with the KM plotter data, a significant association was found between strongly positive nuclear SASH1 expression and favourable breast cancer-specific survival (BCSS) in ER+ breast cancer (p=0.0012; HR 1.4 [1.16-2.69]). SASH1 stratification of outcome in ER+ disease showed similar trends in subgroups with either high or low proliferative indices according to Ki67 expression or mitotic score. The proportions of SASH1-high and -low cases were similar in ER+ subgroups with high or low proliferative activity (FIG. 8C/D). A multivariate Cox regression model in ER+ cases that included HER2 status, Ki67 status, tumour size and histological grade revealed that SASH1 expression was independently associated with BCSS (HR=0.45; 95% confidence interval 0.27-0.77; p=0.0037; Table 4). The trend in the ER-negative cohort was similar to KM analysis of SASH1 mRNA data, though this did not reach statistical significance (p=0.16). Around 5% of cases (n=19/379) exhibited tumour cell cytoplasmic staining but there was no association with the available clinicopathologic parameters studied including survival.

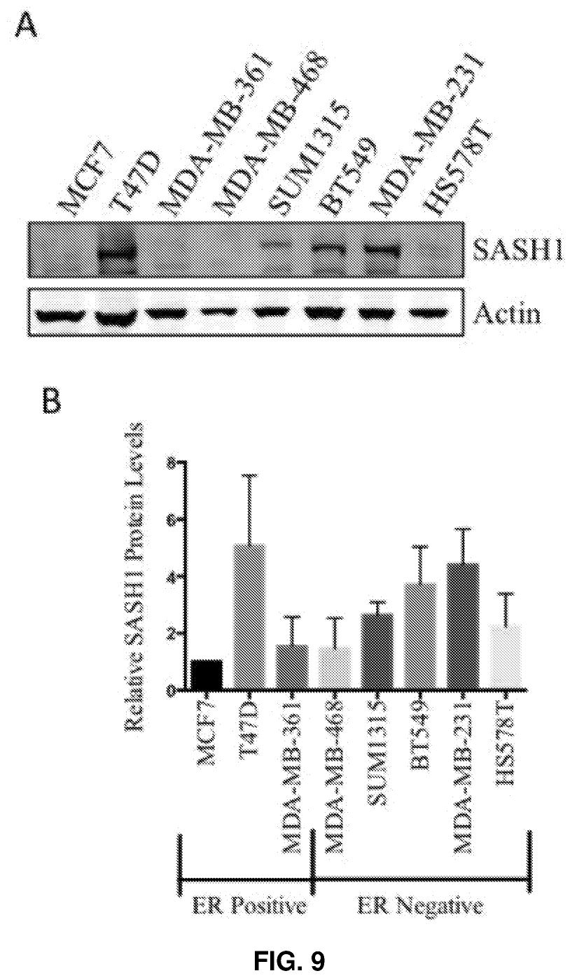

To explore the function of SASH1 in breast cancer, protein expression in three ER-positive and five ER-negative breast cancer cell lines was quantified by immunoblot analysis. This revealed variable expression between cell lines, with three high expressing cell lines, T47-D, BT-549 and MDA-MB-231, two moderately expressing lines, Hs578T and SUM-315 and three low expressing lines MCF7, MDA-MB-361 and MDA-MB-468 (FIG. 9 A-B). There was no obvious association between SASH1 expression and ER status in this panel of cell lines.

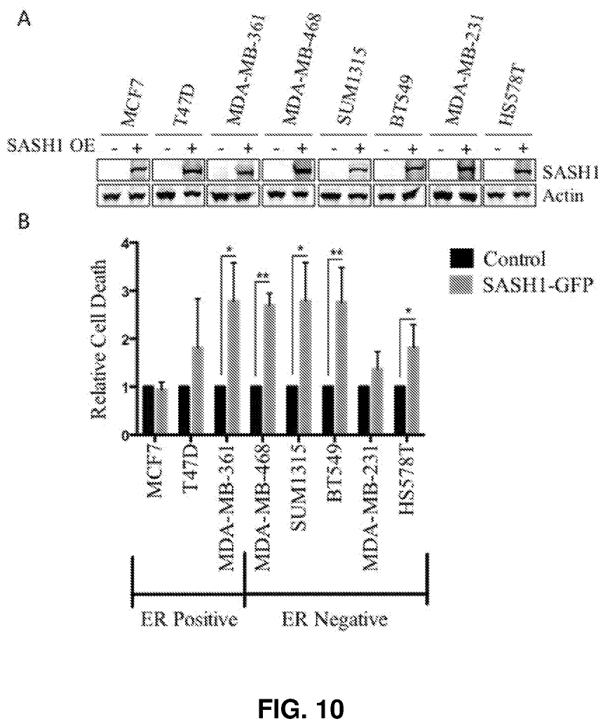

SASH1 has previously been described as a tumour suppressor, with overexpression resulting in an increase in cell death in lung cancer, melanoma, osteosarcoma and glioma cell lines [5, 10-12]. To investigate this a SASH1-GFP fusion protein was transiently over-expressed in breast cancer cell lines. Overexpression resulted in cell death in 7 of the 8 lines tested, significant in 5 lines, with only the caspase 3-deficient MCF7 cells showing no response (FIG. 10).

Hypothesising that increasing SASH1 levels may be a novel approach to cancer therapy, we utilised a connectivity screen using the cmap database (Broad Institute [16]) to identify drugs that lead to induction of SASH1. This identified a direct correlation between chloropyramine treatment and SASH1 mRNA expression (p=0.000005, z-score 2.431). Chloropyramine is a first generation reversible H1-receptor antagonist that is approved in several European countries for management of allergic conditions such as conjunctivitis and bronchial asthma.

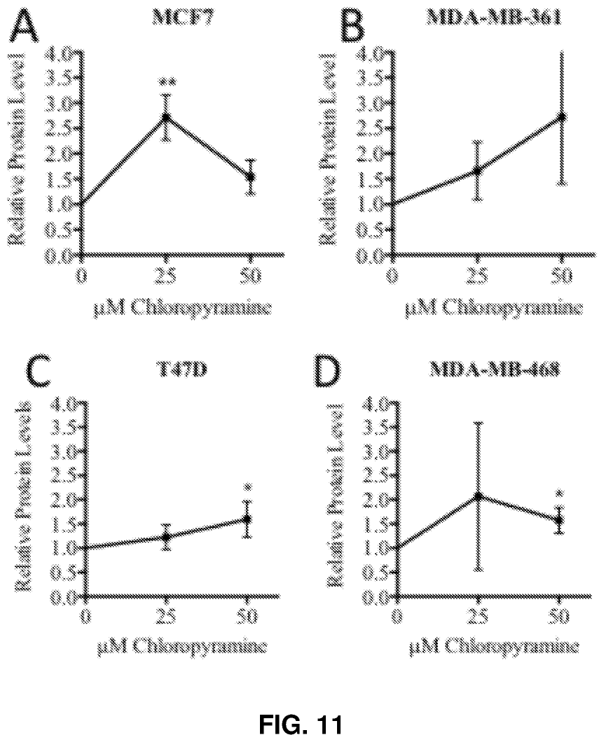

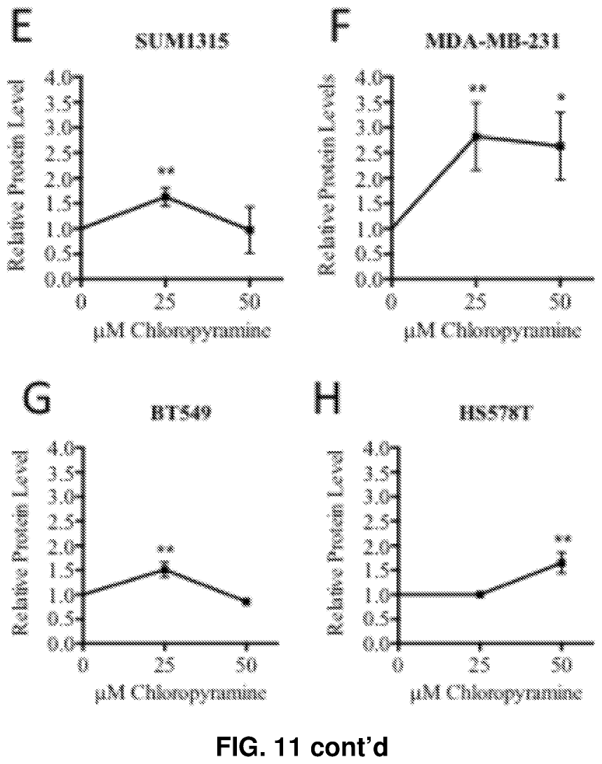

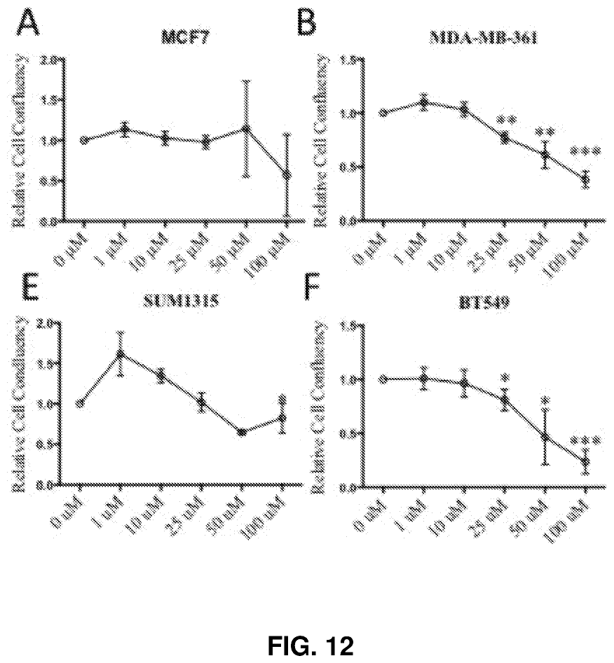

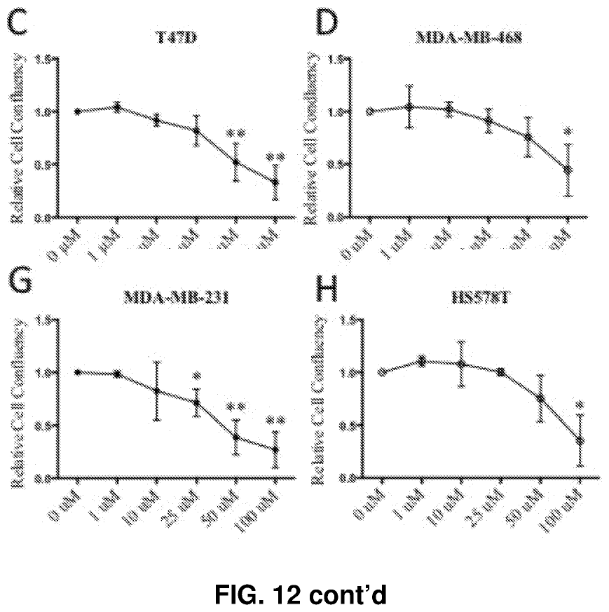

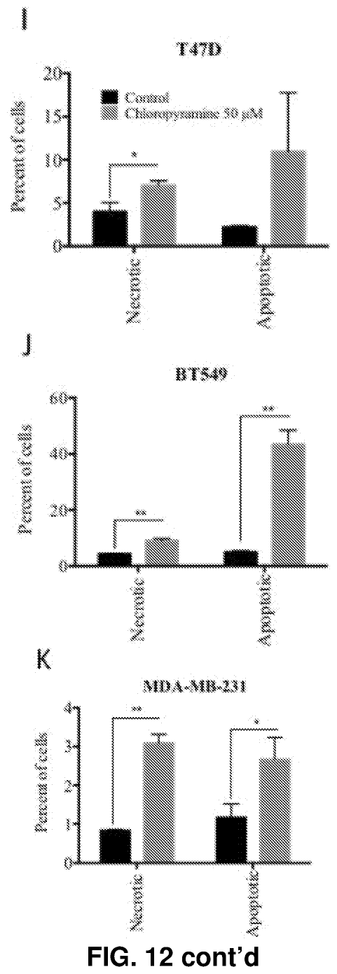

After validating the chloropyramine-mediated induction of SASH1 in breast cancer cell lines at the protein level (FIG. 11), we investigated whether this treatment could mimic the effect of SASH1 over-expression on cell growth and survival. Treatment with chloropyramine inhibited cell growth in 7 of the 8 lines treated (FIG. 12A-H). To investigate whether this was due to induction of apoptosis, we analysed post-treatment levels of Annexin V in the three most sensitive cell lines, T47-D, MDA-MB-231 and BT-549. All three lines exhibited an increase in Annexin V (FIG. 12I-K), indicating induction of apoptosis.

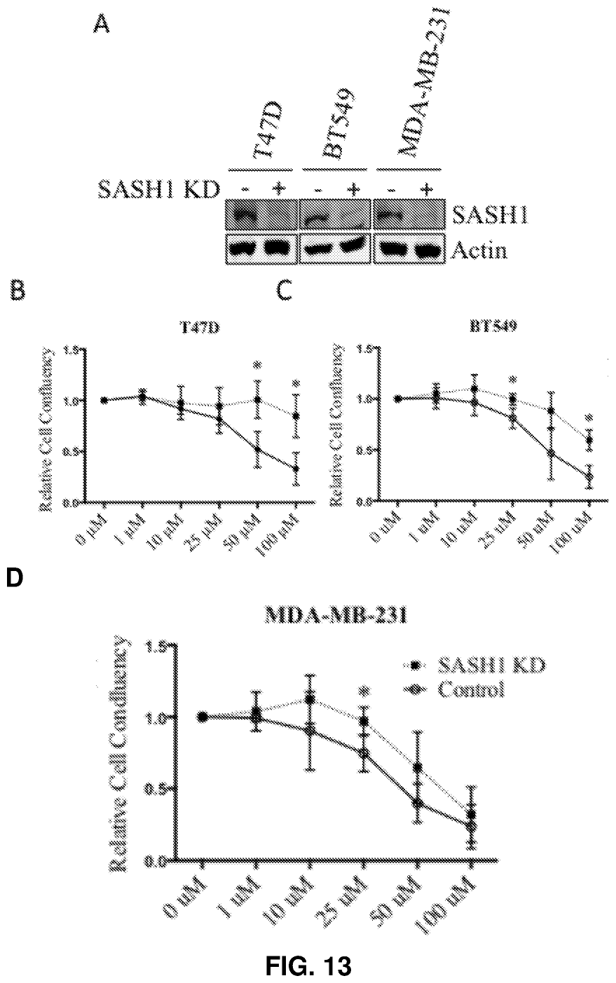

To determine whether the chloropyramine-induced cell death was SASH1-dependent, we transduced T47-D, MDA-MB-231 and BT-549 cells with SASH1-targeted siRNA prior to treatment. This experiment demonstrated that SASH1 depletion partially rescued the cell death response in all three lines (FIG. 13A-D), suggesting chloropyramine-induced cell death is at least in part dependent upon SASH1 function.

TABLE 2
Multivariate Cox regression analysis of factors associated with 15
year breast cancer relapse-free survival in the KM Plotter database
[13]. CI, confidence interval; HR, hazard ratio; MKI67, Ki67 gene.

| | ER positive cases | | | ER negative cases | | |
|---|---|---|---|---|---|---|
| transcript | HR | 95% CI | p value | HR | 95% CI | p value |
| SASH1 | 0.74 | 0.61-0.9 | 0.0032 | 1.89 | 1.24-2.86 | 0.0036 |
| MKI67 | 1.39 | 1.09-1.75 | 0.0068 | 1.02 | 0.71-1.47 | ns |
| ERBB2 | 1.20 | 0.94-1.54 | ns | 1.33 | 0.92-10 | ns |

TABLE 3
Relationships between SASH1 protein expression and clinicopathologic indicators in breast cancer
using the Queensland follow-up (QFU) cohort. p Values shown are from chi square tests.

| | SASH1 | n | | | | % cases | | | |
|---|---|---|---|---|---|---|---|---|---|
| | staining | total | negative | weak-mod | strong | negative | weak-mod | strong | p value |
| Histological | IDC | 228 | 56 | 79 | 93 | 24.6 | 34.6 | 40.8 | ns |
| type | Lobular/variants | 44 | 9 | 17 | 18 | 20.5 | 38.6 | 40.9 | |
| | Mixed | 31 | 8 | 10 | 13 | 25.8 | 32.3 | 41.9 | |
| | ducto-lob | | | | | | | | |
| | Mixed | 34 | 4 | 13 | 17 | 11.8 | 38.2 | 50 | |
| | Metaplastic | 15 | 4 | 6 | 5 | 26.7 | 40 | 33.3 | |
| | Special | 27 | 6 | 13 | 8 | 22.2 | 48.1 | 29.6 | |
| | types | | | | | | | | |
| | n | 379 | | | | | | | |
| Grade | 1 | 51 | 8 | 18 | 25 | 15.7 | 35.3 | 49 | ns |
| | 2 | 182 | 43 | 72 | 67 | 23.6 | 39.6 | 36.8 | |
| | 3 | 146 | 36 | 48 | 62 | 24.7 | 32.9 | 42.5 | |
| | n | 379 | | | | | | | |
| Age | >50 yr | 250 | 54 | 94 | 102 | 21.6 | 37.6 | 40.8 | ns |
| | ≤50 yr | 119 | 27 | 40 | 52 | 22.7 | 33.6 | 43.7 | |
| | n | 369 | | | | | | | |
| Lymph | Negative | 113 | 28 | 38 | 47 | 24.8 | 33.6 | 41.6 | ns |
| node status | Positive | 98 | 23 | 46 | 29 | 23.5 | 46.9 | 29.6 | |
| | n | 211 | | | | | | | |
| Tumour size | <2 cm | 157 | 43 | 49 | 65 | 27.4 | 31.2 | 41.4 | ns |
| | 2-5 cm | 141 | 25 | 57 | 59 | 17.7 | 40.4 | 41.8 | |
| | >5 cm | 29 | 5 | 11 | 13 | 17.2 | 37.9 | 44.8 | |
| | n | 327 | | | | | | | |
| Lymphovascular | Absent | 283 | 66 | 107 | 110 | 23.3 | 37.8 | 38.9 | ns |
| invasion | Present | 95 | 20 | 31 | 44 | 21.1 | 32.6 | 46.3 | |
| | n | 378 | | | | | | | |
| Lymphocytic | Absent | 136 | 34 | 55 | 47 | 25 | 40.4 | 34.6 | ns |
| infiltrate | Mild | 162 | 36 | 53 | 73 | 22.2 | 32.7 | 45.1 | |
| | Moderate- | 80 | 17 | 30 | 33 | 21.3 | 37.5 | 41.3 | |
| | severe | | | | | | | | |
| | n | 378 | | | | | | | |
| Central | Absent | 337 | 77 | 124 | 136 | 22.8 | 36.8 | 40.4 | ns |
| scarring/ | Present | 42 | 10 | 14 | 18 | 23.9 | 33.3 | 42.9 | |
| fibrosis | n | 379 | | | | | | | |
| Tumour | Infiltrative | 324 | 72 | 114 | 138 | 22.2 | 35.2 | 42.6 | ns |
| border | Pushing | 55 | 15 | 24 | 16 | 27.3 | 43.6 | 29.1 | |
| | n | 379 | | | | | | | |
| Ki67 | Low | 307 | 73 | 115 | 119 | 23.8 | 37.5 | 38.8 | ns |
| expression | High | 53 | 10 | 16 | 27 | 18.9 | 30.2 | 50.9 | |
| (20% threshold) | n | 360 | | | | | | | |
| HER2 | Negative | 343 | 81 | 125 | 137 | 23.6 | 36.4 | 39.9 | ns |
| status | Positive | 39 | 7 | 12 | 20 | 17.9 | 30.8 | 51.3 | |
| (CISH) | n | 382 | | | | | | | |
| ER status | Positive | 285 | 58 | 110 | 117 | 20.4 | 38.6 | 41.1 | 0.0035 |
| | Negative | 82 | 28 | 17 | 37 | 34.1 | 20.7 | 45.1 | |
| | n | 367 | | | | | | | |
| TN status | Non-TNBC | 311 | 64 | 115 | 132 | 20.6 | 37 | 42.4 | ns |
| | TNBC | 68 | 22 | 22 | 24 | 32.4 | 32.4 | 35.3 | |
| | n | 379 | | | | | | | |
| Other | HER2+ | 38 | 7 | 12 | 19 | 18.4 | 31.6 | 50 | ns |
| prognostic | HR+/HER2− | 235 | 56 | 88 | 91 | 23.8 | 37.4 | 38.7 | |
| subgroups | neg | | | | | | | | |
| | (Ki67-high) | | | | | | | | |
| | HR+/HER2− | 23 | 1 | 9 | 13 | 4.3 | 39.1 | 56.5 | |
| | neg | | | | | | | | |
| | (Ki67-low) | | | | | | | | |
| | TN | 54 | 18 | 17 | 19 | 33.3 | 31.5 | 35.2 | |
| | (basal- | | | | | | | | |
| | like) | | | | | | | | |
| | TN | 12 | 3 | 5 | 4 | 25 | 41.7 | 33.3 | |
| | (non- | | | | | | | | |
| | basal) | | | | | | | | |
| | n | 362 | | | | | | | |

TABLE 4
Prognostic value of nuclear SASH1 expression in ER-positive breast
cancer over 25 years. Univariate (Kaplan Meier; a) and multivariate
(stepwise Cox proportional hazards regression; b) analysis of
factors associated with breast cancer-specific survival among
223 ER+ cases in the Queensland Follow-up Cohort.

| | Univariate^{a} | | | Multivariate^{b} | | |
|---|---|---|---|---|---|---|
| Variables | HR | 95% CI | p value | HR | 95% CI | p value |
| HER2 | 3.23 | 1.29-8.07 | <0.0001 | 2.45 | 1.21-4.98 | 0.0134 |
| Ki67 | 1.87 | 0.88-3.95 | 0.0325 | 2.38 | 1.88-4.76 | 0.0150 |
| SASH1 | 0.54 | 0.35-0.82 | 0.0068 | 0.45 | 0.27-0.77 | 0.0037 |
| Size | 2.32 | 1.52-3.53 | 0.0002 | 1.97 | 1.97-3.22 | 0.0078 |
| Grade | 3.17 | 1.75-5.76 | 0.0085 | — | — | ns |

### DISCUSSION

Based on its association with favourable prognosis in several human malignancies [4-6, 10-12, 19, 20] and evidence of adverse effects on cancer cell line viability in vitro [5, 10-12], SASH1 has been proposed as a tumour suppressor [4-7, 10, 12, 21, 22]. Though mechanistic studies are generally lacking at present, there is some evidence suggesting that SASH1 can suppress PI3K and Akt signalling [23]. The prognostic significance in breast cancer had been less well characterised than other malignancies, so we investigated this using two well-annotated clinical sample cohorts. While overall we found that SASH1 was associated with favourable prognosis, stratifying the cases based on ER status revealed that this was driven by the more prevalent ER-positive cases (75-80% of the cohorts analysed). In fact SASH1 associated with poor outcome in ER-negative breast cancer. Although SASH1 has been coined a tumour suppressor, the opposing data from ER-positive and -negative breast cancer suggest that this may be oversimplifying its role and that context is critical. Indeed, interrogating other KM Plotter cancer datasets we found strong associations between SASH1 mRNA expression and better overall survival in lung cancer but very poor outcome in gastric cancer (Data not shown).

The study found no obvious association between SASH1 expression and ER status in a panel of eight breast cancer cell lines. Furthermore ectopic expression of SASH1 reduced cell viability independent of ER status. Collectively these observations suggested that in some contexts, SASH1 suppression could reflect a process required for cancer cell viability and aggressive clinical behaviour, and hypothesised that increasing its expression could be a novel treatment strategy. To identify drug candidates with this capability, we performed in silico connectivity mapping using the cmap database (Broad Insitute [16]) and identified the antihistamine chloropyramine as a candidate SASH1 inducer. Others have shown that chloropyramine reduces survival of cell lines from melanoma, neuroblastoma, breast and pancreatic cancers, possibly involving inhibition of FAK and VEGFR3 signalling [17, 18, 24-26]. Consistent with the connectivity screen, chloropyramine induced SASH1 expression in 7 of the 8 breast cancer cell lines tested, and reduced the viability of 6 of 8. Transducing the three most sensitive lines with SASH1 siRNA prior to treatment partially rescued the cytotoxic response, suggesting that chloropyramine-induced cancer cell line death is at least partly mediated by SASH1. There is at least one existing report suggesting that chloropyramine can reduce breast cancer xenograft growth in vivo [18], though additional preclinical studies are required to more comprehensively characterise the anti-tumour activity of the agent.

Breast cancer management has improved substantially over the last few decades, but in Australia, the US and UK, 15-20% of patients still do not survive 10 years after diagnosis [27, 28]. This amounts to a large proportion of cancer-related morbidity and mortality and cost to the public health sector. Tumours that do not respond to current first-line therapies are likely to be more complex and heterogeneous. Disease control in the future will depend on an increased understanding of the molecular biology of the disease, leading to identification of novel personalised medicine therapy approaches linked to companion diagnostics. In silico connectivity screening provides a means to fast-track the identification of gene-drug associations and drug repurposing opportunities. In silico mapping of associations between induction of SASH1 and ‘off-the-shelf’ drugs identified a novel candidate, chloropyramine, with antitumour activity in vitro. Chloropyramine and other first-generation H1 antagonists are sedating because they cross the blood-brain-barrier, and were therefore superseded by peripherally-acting agents for the treatment of allergy. Given that brain uptake can be desirable in molecular oncology and chloropyramine is otherwise well-tolerated, the potential application for this or structurally related agents for low toxicity treatment of breast and other cancers deserves further mechanistic and preclinical investigation. Furthermore phase 0 and dose finding phase I, neoadjuvant, biomarker driven clinical trials in patients with breast cancer could allow us to confirm the pharmacodynamic induction of SASH1 by chloropyramine that would underpin further repurposing studies of the agent in the future. This is particularly relevant in triple negative breast cancer where therapeutic options are currently limited but also in ER and HER2 disease that has become resistant to current therapeutic approaches.

### REFERENCES

1. Breast Cancer. Prevention and Control [http://www.who.int/cancer/detection/breastcancer/en/indexl.html]
2. Davis S L, Eckhardt S G, Tentler J J, Diamond J R. Triple-negative breast cancer: bridging the gap from cancer genomics to predictive biomarkers. Ther Adv Med Oncol. 2014; 6: 88-100.
3. Anders C K, Carey L A. Biology, Metastatic Patterns, and Treatment of Patients with Triple-Negative Breast Cancer. Clinical Breast Cancer. 2009; 9: S73-S81.
4. Zeller C, Hinzmann B, Seitz S, Prokoph H, Burkhard-Goettges E, Fischer J, Jandrig B, Schwarz L E, Rosenthal A, Scherneck S. SASH1: a candidate tumor suppressor gene on chromosome 6q24.3 is downregulated in breast cancer. Oncogene. 2003; 22:2972-2983.
5. Chen E G, Chen Y, Dong L L, Zhang J S. Effects of SASH1 on lung cancer cell proliferation, apoptosis, and invasion in vitro. Tumour Biol. 2012.
6. Rimkus C, Martini M, Friederichs J, Rosenberg R, Doll D, Siewert J R, Holzmann B, Janssen K P. Prognostic significance of downregulated expression of the candidate tumour suppressor gene SASH1 in colon cancer. Br J Cancer. 2006; 95: 1419-1423.
7. Sheyu L, Hui L, Junyu Z, Jiawei X, Honglian W, Qing S, Hengwei Z, Xuhui G, Qinghe X, Lin H. Promoter methylation assay of SASH1 gene in breast cancer. J BUON. 2013; 18:891-898.
8. Koch C A, Anderson D, Moran M F, Ellis C, Pawson T. SH2 and SH3 domains. elements that control interactions of cytoplasmic signaling proteins. Science. 1991; 252: 668-674.
9. Claudio J O, Zhu Y X, Benn S J, Shukla A H, McGlade C J, Falcioni N, Stewart A K. HACS1 encodes a novel SH3-SAM adaptor protein differentially expressed in normal and malignant hematopoietic cells. Oncogene. 2001; 20: 5373-5377.
10. Meng Q, Zheng M, Liu H, Song C, Zhang W, Yan J, Qin L, Liu X. SASH1 regulates proliferation, apoptosis, and invasion of osteosarcoma cell. Mol Cell Biochem. 2013; 373:201-210.
11. Lin S, Zhang J, Xu J, Wang H, Sang Q, Xing Q, He L. Effects of SASH1 on melanoma cell proliferation and apoptosis in vitro. Mol Med Rep. 2012; 6: 1243-1248.
12. Yang L, Liu M, Gu Z, Chen J, Yan Y, Li J. Overexpression of SASH1 related to the decreased invasion ability of human glioma U251 cells. Tumour Biol. 2012.
13. Gyorffy B, Lanczky A, Eklund A C, Denkert C, Budczies J, Li Q, Szallasi Z. An online survival analysis tool to rapidly assess the effect of 22,277 genes on breast cancer prognosis using microarray data of 1,809 patients. Breast Cancer Res Treat. 2010; 123: 725-731.
14. Al-Ejeh F, Simpson P T, Saunus J M, Klein K, Kalimutho M, Shi W, Miranda M, Kutasovic J, Raghavendra A, Madore J, Reid L, Krause L, Chenevix-Trench G, Lakhani S R, Khanna K K. Meta-analysis of the global gene expression profile of triple-negative breast cancer identifies genes for the prognostication and treatment of aggressive breast cancer. Oncogenesis. 2014; 3: e124.
15. Junankar S, Baker L A, Roden D L, Nair R, Elsworth B, Gallego-Ortega D, Lacaze P, Cazet A, Nikolic I, Teo W S, Yang J, McFarland A, Harvey K, Naylor M J, Lakhani S R, Simpson P T, Raghavendra A, Saunus J, Madore J, Kaplan W, Ormandy C, Millar EKA, O'Toole S, Yun K, Swarbrick A. ID4 controls mammary stem cells and marks breast cancers with a stem cell-like phenotype. Nat Comms. 2015; 6: 6548.
16. Lamb J, Crawford E D, Peck D, Modell J W, Blat I C, Wrobel M J, Lerner J, Brunet J-P, Subramanian A, Ross K N, Reich M, Hieronymus H, Wei G, Armstrong S A, Haggarty S J, Clemons P A, Wei R, Carr S A, Lander E S, Golub T R. The Connectivity Map. using geneexpression signatures to connect small molecules, genes, and disease. Science. 2006; 313:1929-1935.
17. Kurenova E, Ucar D, Liao J, Yemma M, Gogate P, Bshara W, Sunar U, Seshadri M, Hochwald S N, Cance W G. A FAK scaffold inhibitor disrupts FAK and VEGFR-3 signaling and blocks melanoma growth by targeting both tumor and endothelial cells. Cell Cycle. 2014; 13: 2542-2553.
18. Kurenova E V, Hunt D L, He D, Magis A T, Ostrov D A, Cance W G. Small molecule chloropyramine hydrochloride (C4) targets the binding site of focal adhesion kinase and vascular endothelial growth factor receptor 3 and suppresses breast cancer growth in vivo. J Med Chem. 2009; 52: 4716-4724.
19. Zhou D, Wei Z, Wang T, Zai M, Guo L, He L, Xing Q. SASH1 regulates melanocyte transepithelial migration through a novel Galphas-SASH1-IQGAP1-E-Cadherin dependent pathway. Cell Signal; 2013.
20. Yang L, Zhang H, Yao Q, Yan Y, Wu R, Liu M. Clinical Significance of SASH1 Expression in Glioma. Dis Markers. 2015, 2015. 383046.
21. Nitsche U, Rosenberg R, Balmert A, Schuster T, Slotta-Huspenina J, Herrmann P, Bader F G, Friess H, Schlag P M, Stein U, Janssen K-P. Integrative marker analysis allows risk assessment for metastasis in stage II colon cancer. Ann Surg. 2012; 256: 763-71-discussion 771.
22. Peng L, Wei H, Liren L. Promoter methylation assay of SASH1 gene in hepatocellular carcinoma. J BUON. 2014; 19: 1041-1047.
23. Sun D, Zhou R, Liu H, Sun W, Dong A, Zhang H. SASH1 inhibits proliferation and invasion of thyroid cancer cells through PI3K/Akt signaling pathway. Int J Clin Exp Pathol. 2015; 8: 12276-12283.
24. Gogate P N, Ethirajan M, Kurenova E V, Magis A T, Pandey R K, Cance W G. Design, synthesis, and biological evaluation of novel FAK scaffold inhibitors targeting the FAKVEGFR3 protein-protein interaction. Eur J Med Chem. 2014; 80: 154-166.
25. Stewart J E, Ma X, Megison M, Nabers H, Cance W G, Kurenova E V, Beierle E A. Inhibition of FAK and VEGFR-3 binding decreases tumorigenicity in neuroblastoma. Molecular carcinogenesis. 2015; 54: 9-23.
26. Kurenova E, Liao J, He D-H, Hunt D, Yemma M, Bshara W, Seshadri M, Cance W G. The FAK scaffold inhibitor C4 disrupts FAK-VEGFR-3 signaling and inhibits pancreatic cancer growth. Oncotarget. 2013; 4: 1632-1646.
27. Cancer Research UK [http.//www.cancerresearchuk.org/health-professional/breastcancer-statistics]
28. Cancer Australia Annual Report 2013-2014 [https.//canceraustralia.gov.au/publicationsand-resources/cancer-australia-publicanons/cancer-australia-annual-report-2013-2014]
29. Li Q, Birkbak N J, Gyorffy B, Szallasi Z, Eklund A C. Jetset. selecting the optimal microarray probe set to represent a gene. BMC Bioinformatics. 2011; 12: 474.
30. Field S, Uyttenhove C, Stroobant V, Cheou P, Donckers D, Coutelier J-P, Simpson P T, Cummings M C, Saunus J M, Reid L E, Kutasovic J R, McNicol A M, Kim B R, Kim J H, Lakhani S R, Neville A M, Van Snick J, Jat P S. Novel highly specific anti-periostin antibodies uncover the functional importance of the fascilin 1-1 domain and highlight preferential expression of periostin in aggressive breast cancer. International journal of cancer Journal international du cancer. 2015.
31. Bolderson E, Petermann E, Croft L, Suraweera A, Pandita R K, Pandita T K, Helleday T, Khanna K K, Richard D J. Human single-stranded DNA binding protein 1 (hSSB1/NABP2) is required for the stability and repair of stalled replication forks. Nucleic Acids Research. 2014; 42: 6326-6336.
32. Bolderson E, Richard D J, Zhou B B, Khanna K K. Recent advances in cancer therapy targeting proteins involved in DNA double-strand break repair. Clin Cancer Res. 2009; 15:6314-6320.
33. Zhang S-D, Gant T W. sscMap. an extensible Java application for connecting smallmolecule drugs using gene-expression signatures. BMC Bioinformatics. 2009; 10: 236.
34. Zhang S-D, Gant T W. A simple and robust method for connecting small-molecule drugs using gene-expression signatures. BMC Bioinformatics. 2008; 9: 258.

### Example 3

In this study the relationship between SASH1 protein levels and sensitivity of ovarian cancer tumour samples to PARP inhibitors was investigated using PDX tumour samples from ovarian cancer patients.

### Materials and Methods

PDX tissue from 12 patients with high grade ovarian cancer and known response to rucaparib were received from the Walter and Eliza Hall Institute. These samples are described more details in Kondrashova et al., [1]. 15 mg of tissue from each sample was lysed in 4004 of RIPA buffer with protease and phosphatase inhibitor (Invitrogen). Samples were homogenised and then sonicated with cellular debris removed through centrifugation. A BCA assay was performed to calculate protein concentration with 40 μg run on a gel and a western blot performed as previously described. SASH1 protein levels were quantified with ImageJ and normalised against γ-Tubulin.

### Results

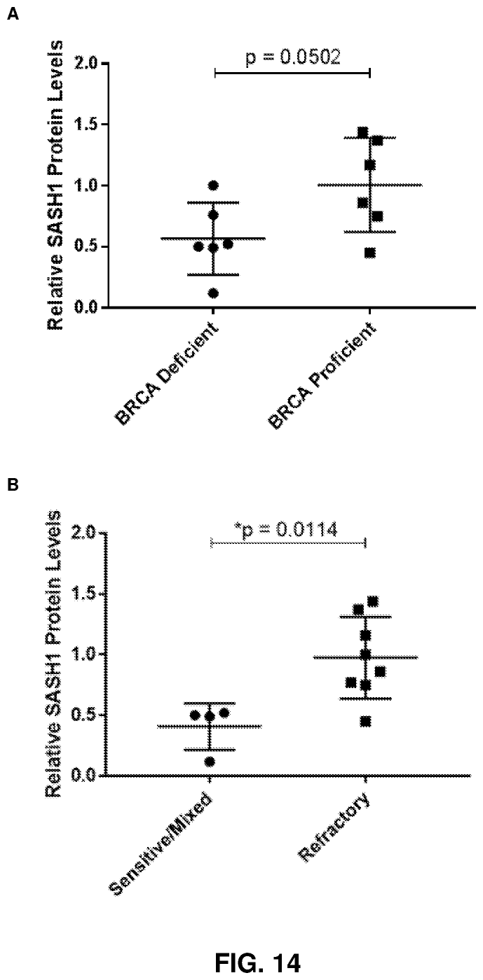

SASH1 protein levels were generally lower in BRCA deficient and higher in BRCA proficient tumours although there is still a noticeable overlap in SASH1 levels between these two groups (FIG. 14 A). By contrast SASH1 levels provided a far stronger and more highly significant prediction of responsiveness to treatment with rucaparib whereby lower SASH1 levels were found in patients with a sensitive or mixed response while higher levels were found in patients who were refractory (FIG. 14 B).

### DISCUSSION

These results demonstrate the ability of SASH1 levels to predict patient and tumour sensitivity to PARP inhibitors and such information will help to stratify patients into treatment groups and improve outcomes at the clinic. These results also show that SASH1 levels may be measured in patients to greatly improve the efficiency and lower the costs of conducting clinical trials to develop new PARP inhibitors by allowing clinicians to identify those patients who are more likely to respond to PARP inhibition.

### REFERENCES

1. Olga Kondrashova, Monique Topp, et al, Methylation of all BRCA1 copies predicts response to the PARP inhibitor rucaparib in ovarian carcinoma. Nature Communications volume 9, Article number: 3970 (2018).

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

1.-31. (canceled)

32. A method of treating a cancer in a subject, said method comprising the step of determining an expression level of a SASH1 nucleic acid or protein in one or a plurality of cancer cells, tissues or organs of the subject and based on the determination made, initiating, continuing, modifying or discontinuing a cancer treatment.

33. The method of claim 32, wherein the cancer treatment comprises the administration of a therapeutically effective amount of an anti-cancer agent that inhibits activity of an enzyme that mediates repair of a DNA strand break.

34. The method of claim 33, wherein the cancer treatment comprises administering to the subject a therapeutically effective amount of an agent that inhibits or prevents the expression and/or an activity of SASH1.

35. The method of claim 33, wherein the enzyme is poly(ADP-ribose) polymerase (PARP).

36. The method of claim 33, wherein the anti-cancer agent is or comprises a PARP inhibitor.

37. The method of claim 36, wherein the PARP inhibitor is selected from one or more of the group consisting of olaparib, veliparib, rucaparib, iniparib, talazoparib, niraparib, 3-aminobenzamide, ME0328, PJ34, AG-14361, INO-1001, UPF-1069, AZD-2461, CEP 9722, and A-966492.

38. A method of treating a cancer in a subject, comprising the step of administering to the subject a therapeutically effective amount of an agent that inhibits or prevents the expression and/or an activity of SASH1 in combination with an anti-cancer agent that inhibits activity of an enzyme that mediates repair of a DNA strand break.

39. The method of claim 38, wherein the enzyme is poly(ADP-ribose) polymerase (PARP).

40. The method of claim 38, wherein the anti-cancer agent is or comprises a PARP inhibitor.

41. The method of claim 40, wherein the PARP inhibitor is selected from one or more of the group consisting of olaparib, veliparib, rucaparib, iniparib, talazoparib, niraparib, 3-aminobenzamide, ME0328, PJ34, AG-14361, INO-1001, UPF-1069, AZD-2461, CEP 9722, and A-966492.

42. A method of treating a cancer in a subject, comprising the step of administering to the subject a therapeutically effective amount of an agent that increases the expression and/or an activity of SASH1.

43. The method of claim 42, wherein the agent is a small organic molecule.

44. A method of identifying an agent useful in the treatment of a cancer in a subject, comprising the steps of:
(a) contacting a cell that expresses a SASH1 protein or nucleic acid with a candidate agent; and
(b) determining whether the candidate agent modulates the expression and/or an activity of SASH1,
wherein if the candidate agent modulates the expression and/or activity of SASH1 then that candidate agent has been identified as an agent useful for the treatment of cancer.

45. The method of claim 44, wherein the candidate agent, at least partly, reduces, eliminates, suppresses or inhibits the expression and/or the activity of SASH1.

46. The method of claim 44, wherein the candidate agent, at least partly, increases the expression and/or the activity of SASH1.

47. The method of claim 44, wherein the candidate agent is selected from the group consisting of an antibody and a small organic molecule.

48. The method of claim 44, wherein the cancer is a cancer of the reproductive system.

49. The method of claim 48, wherein the cancer of the reproductive system is selected from the group consisting of breast cancer, ovarian cancer, cervical cancer, uterine cancer, prostate cancer and testicular cancer.

50. The method of claim 48, wherein the cancer of the reproductive system is selected from the group consisting of breast cancer and lung cancer.

51. A method of predicting the responsiveness of a cancer to an anti-cancer agent in a subject and, optionally, treating the subject, wherein the anti-cancer agent inhibits activity of an enzyme that mediates repair of a DNA strand break, said method comprising the step of:
determining an expression level of a SASH1 nucleic acid or protein in one or a plurality of cancer cells, tissues or organs of the subject, wherein an expression level of the SASH1 nucleic acid or protein indicates or correlates with relatively increased or decreased responsiveness of the cancer to the anti-cancer agent; and
optionally, treating the cancer in the subject.

52. The method of claim 51, wherein the enzyme is poly(ADP-ribose) polymerase (PARP).

53. The method of claim 51, wherein the anti-cancer agent is or comprises a PARP inhibitor.

54. The method of claim 53, wherein the PARP inhibitor is selected from one or more of the group consisting of olaparib, veliparib, rucaparib, iniparib, talazoparib, niraparib, 3-aminobenzamide, ME0328, PJ34, AG-14361, INO-1001, UPF-1069, AZD-2461, CEP 9722, and A-966492.

55. The method of claim 52 or 53, wherein a decreased level of a SASH1 nucleic acid or protein indicates or correlates with relatively increased responsiveness of the cancer to the PARP inhibitor and/or an increased level of a SASH1 nucleic acid or protein indicates or correlates with relatively decreased responsiveness of the cancer to the PARP inhibitor.

56. A method of determining a prognosis for a breast cancer in a subject and, optionally, treating the subject, said method comprising the step of:
determining an expression level of SASH1 nucleic acid or protein in one or a plurality of cancer cells, tissues or organs of the subject, wherein a modulated expression level of SASH1 indicates or correlates with a less or more favorable cancer prognosis for said breast cancer; and
optionally, treating the cancer in the subject.

57. The method of claim 56, wherein the breast cancer is ER positive (ER⁺) breast cancer or ER negative (ER−) breast cancer.

58. A kit for predicting responsiveness of a cancer to an anti-cancer agent in a subject, wherein the anti-cancer agent inhibits activity of an enzyme that mediates repair of a DNA strand break, the kit comprising at least one reagent capable of determining an expression level of a SASH1 protein or encoding nucleic acid in one or a plurality of cancer cells, tissues or organs of the subject, wherein the expression level of the SASH1 protein or encoding nucleic acid indicates or correlates with relatively increased or decreased responsiveness of the cancer to the anti-cancer agent.
